(19) United States
(12) Patent Application Publication
(10) Pub. No.: US 2017/0035791 A1
(43) Pub. Date: Feb. 9, 2017

(54) COMBINATION OF BETA-GLUCOSYLCERAMIDE AND POLYETHOXYLATED CASTOR OIL AND OTHER ADJUVANTS FOR CONTROLING BLOOD SUGAR LEVELS, IMMUNOPROTECTION AND HEPATOPROTECTION

(71) Applicant: NATURAL SHIELD ISRAEL 2016 LTD, Kfar Tavor (IL)

(72) Inventor: Yaron ILAN, Kfar Tavor (IL)

(73) Assignee: NATURAL SHIELD ISRAEL 2016 LTD, Kfar Tavor (IL)

(21) Appl. No.: 15/304,408

(22) PCT Filed: Apr. 14, 2014
(86) PCT No.: PCT/IL2015/050401

§ 371 Date: Oct. 14, 2016

Related U.S. Application Data
(60) Provisional application No. 61/979,112, filed on Apr. 14, 2014.

(51) Int. Cl. A61K 31/7028; A23L 2/60; A23L 33/10; A61K 36/47; A61K 45/06

(52) U.S. Cl. CPC A61K 31/7028; A61K 36/47; A61K 45/06; A23L 33/10; A23L 2/60; A23V 2002/00

(57) ABSTRACT

The disclosure relates to compositions and methods for adding beta glucosylceramide and/or Cremophor EL (CE), or an adjuvant selected from polyethylene glycol and beta cyclo dextrin or any type of beta or alpha glucosylceramide with/without CE, or CE alone, to drinks or foods to serve as liver protectors, sugar protectors, and anti inflammatory protectors.

### FIELD OF INVENTION

The present invention relates to the field of food supplements and therapeutic compositions. More particularly, the invention relates to combined therapeutic compositions and food supplements comprising GC and polyethoxylated castor oil (e.g. Cremophore EL) and optionally other adjutants such as polyethylene glycol or beta cyclo dextrin for controlling blood sugar levels and prevention of symptoms of pathological conditions related thereto.

### BACKGROUND REFERENCES

1. Ludwig D S, et al. Relation between consumption of sugar-sweetened drinks and childhood obesity: a prospective, observational analysis. The Lancet, Volume 357, Issue 9255, pages 505-508 (2001).
2. Schulze M B, et al. Sugar-sweetened beverages, weight gain, and incidence of type 2 diabetes in young and middle-aged women. JAMA, Volume 292, Issue 8, pages 927-934 (2004).
3. Dhingra R et al. Soft drink consumption and risk of developing cardiometabolic risk factors and the metabolic syndrome in middle-aged adults in the community. Circulation, Volume 116, Issue 5, pages 480-488 (2007).
4. Global Burden of Disease 2010 http://rt.com/usa/soda-sugar-danger-research-singh-559
5. CDC Facts Sheets http://www.cdc.gov/alcohol/fact-sheets/alcohol-use.htm
6. Swift R and Davidson D. Alcohol hangover: mechanisms and mediators. Alcohol Health & Research World, Volume 22, Issue 1, pages 54-60 (1998)
7. Menon K V et al. Pathogenesis, diagnosis, and treatment of alcoholic liver disease. Mayo Clinic Proceedings, Volume 76, Issue 10, pages 1021-1029 (2001).
8. O'Shea R S et al. Alcoholic liver disease: AASLD Practice Guidelines. Hepatology, Volume 51, Issue 1, pages 307-328 (2010).
9. Morgan M Y. The prognosis and outcome of alcoholic liver disease. Alcohol and Alcoholism, Volume 2 (Suppl), pages 335-343 (1994).
10. Erickson S K. Nonalcoholic fatty liver disease. J Lipid Res, Volume 50 (Suppl), pages S412-S416 (2009).

### BACKGROUND OF THE INVENTION

Stability of the level of blood glucose (or blood sugar) is the basic prerequisite for maintenance of controlled influx and availability of glucose to the cells. Glucose, being the preferential source of energy in virtually all body cells, is essential for normal function of all body systems, which is why blood glucose levels are tightly regulated as a part of metabolic homeostasis governed by pancreatically produced insulin/glucagon feedback. In all vertebrates, regardless of large fluctuations in physical activity and food intake, blood sugar levels are held within very narrow limits. In humans, the normal blood glucose levels (tested while fasting) for non-diabetics, are on average between 70-100 milligrams per deciliter (mg/dL). Blood glucose levels outside the normal range, i.e. persistent hyper- or hypo-glycemia, may be an indicator of a number of medical conditions. Diabetes mellitus characterized by persistent hyperglycemia and is the most prominent disease related to failure of blood sugar regulation. Intake of alcohol causes an initial surge in blood sugar, and later tends to cause hypoglycemia.

Apart from issues of lifestyle and self esteem, controlling blood sugar levels and maintaining a healthy weight are vital to lower the risk of diseases such as type 2 diabetes (the most common adult form of diabetes resulting from insulin resistance), morbid obesity, heart disease, liver disease and cancer. Consequences of chronic alcohol consumption are numerous, apart from risks of injuries sustained in car accidents and liver cirrhosis, there are also risks of anemia, cardiovascular disease, cancer and distinct neurologic and psychiatric disorders. Sugar enriched foods, particularly soft drinks, and alcohol are considered among major health hazards produced by a modern way of living. The Gallup's Annual Consumption Habits Poll conducted in Jul. 9-12, 2012 in the US, for example, indicated that about half of all Americans, 48%, consume on average at least one glass of a soda per day and 66%—over four alcoholic drinks per week.

According to the American Heart Association, soft drinks and other sugar-sweetened beverages (SSBs) are the primary source of added sugars in Americans' diets; their increased consumption has been associated with rising obesity rates. Consumption of SSBs has increased 500% in the past fifty years and is now the single largest category of caloric intake in children, about 10-15% of the total daily calorie intake. The rising prevalence of obesity in children has been linked, in part, to the consumption of SSBs [1]. Consumption of excessive calories and large amounts of rapidly absorbable sugars through SSBs was recognized as one of significant contributors to weight gain and incidence of type 2 diabetes in American women between 1991 to 1999 [2]. In fact, individuals consuming one or more SSB per day have higher odds for developing metabolic syndrome (odds ratio OR=1.48), obesity (OR=1.31), increased waist circumference (OR=1.30), impaired fasting glucose (OR=1.25), higher blood pressure (OR=1.18), hyper-triglyceridemia (OR=1.25), and low high-density lipoprotein cholesterol (OR=1.32) [3]. A recent research by the Harvard School of Public Health summarizing data for Global Burden of Disease for 2010, suggested that SSBs were directly responsible for 133,000 deaths from diabetes, 44,000 deaths from cardiovascular disease and 6,000 deaths from cancer worldwide and for a total of 25,000 deaths in the US alone [4].

Concerns with regard to excessive sugar and alcohol consumption imposed by the modern life style are clear. Public health policy makers and professionals are currently conducting a number of policies to control consumption, including taxation and legislation. The food and beverage industry is increasingly replacing sugary products with sugar-free or artificially sweetened versions. There is however apparent shortage of candidate food additives, natural or synthetic, having potential to counter-balance the negative effects of both, excess sugar and alcohol. Two food additives getting lately a lot of attention as blood sugar busting components are vinegar and cinnamon.

According to the Centers for Disease Control (CDC) statistics for 2006-2010, there are annually 88,000 deaths attributable to excessive alcohol consumption in the US alone, making alcohol the 3^{rd} leading lifestyle-related cause of death in the nation. In US 2006, for example, there were more than 1.2 million emergency room visits and 2.7 million physician office visits due to excessive drinking [5]. As previously mentioned, excessive alcohol consumption has immediate effects on many harmful health conditions, such as in increasing risk of injuries, violence, risky sexual behavior and unprotected sex, miscarriage and stillbirth among pregnant women. Over time, excessive alcohol use can lead to the development of chronic diseases, including liver disease, alcoholic hepatitis and cirrhosis, the latter are among the leading causes of deaths in the US. Long-term health risks also include, but are not limited to, neurological impairments, cardiovascular problems, and psychiatric and social problems.

Several alterations in the metabolic state of the liver and other organs occur in response to the presence of alcohol (ethanol) in the body and can result in low blood sugar levels (hypoglycemia) [6]. Alcohol metabolism leads to a fatty liver and buildup of an intermediate metabolic product, lactic acid, in body fluids (lactic acidosis). Both of these effects can inhibit glucose production. Alcohol-induced hypoglycemia generally occurs after prolonged alcohol consumption coupled with poor nutritional intake, which not only decreases glucose production but also exhaust the reserves of glucose stored in the liver in the form of glycogen, thereby leading to hypoglycemia. Because glucose is the primary energy source of the brain, hypoglycemia can contribute to hangover symptoms such as fatigue, weakness, and mood disturbances. Diabetics are particularly sensitive to the alcohol-induced alterations in blood glucose.

Excessive alcohol consumption is the major cause of liver disease; 15-20% of chronic heavy drinkers develop hepatitis or cirrhosis that can occur concomitantly or in succession. While genetic factors may contribute both to alcoholism and to alcoholic liver disease, malnutrition, particularly vitamin A and E deficiencies, can worsen alcohol-induced liver damage by preventing hepatocyte regeneration [7]. Women are twice as susceptible to alcohol-related liver disease, and may develop alcoholic liver disease with shorter durations and doses of chronic consumption. Alcoholic liver disease evolves as a result of secretion of pro-inflammatory cytokines, oxidative stress, lipid peroxidation and acetaldehyde toxicity ensuing in response to alcohol consumption. These factors cause inflammation, apoptosis and eventually fibrosis of liver cells [8].

Alcoholic liver disease evolves from fatty change through alcoholic hepatitis to alcoholic cirrhosis. Its development is associated with an excess mortality both in relation to the presence of liver disease and to other complications of alcohol abuse. In the majority of patients fatty liver is a benign lesion, which will reverse completely following abstinence from alcohol. Continued drinking is associated with the eventual development of cirrhosis in approximately 20% of individuals. Alcoholic hepatitis is a precirrhotic lesion; progression to cirrhosis is observed more commonly in women, in individuals with severe disease and in those who continue to drink. Thirty-day mortality rates of less than 20% are observed in patients with mild to moderate disease but exceed 40% in individuals with severe liver injury. Survival is significantly reduced in women and in the elderly and is adversely affected by the presence of severe liver injury, evolution to cirrhosis and continued drinking Two-thirds of patients with alcoholic cirrhosis present with decompensated disease; 15% will develop hepatocellular carcinoma. Survival is adversely affected by the presence of decompensated disease, superimposed alcoholic hepatitis, continued drinking and the development of hepatocellular carcinoma [9].

Nonalcoholic fatty liver disease (NAFLD) is rapidly becoming a worldwide public health problem. It is the most common liver disease in the US and, indeed, worldwide. Current estimates are that ˜20% of the general US population has NAFLD. The prevalence in the morbidly obese population has been estimated as 75-92%, while that in the pediatric population as 13-14%. At present, it is estimated that ˜6 million individuals in the US general population have progressed to nonalcoholic steatohepatitis (NASH) and ˜600,000 to NAFLD-related cirrhosis. Thus, the number of individuals at risk for end-stage liver disease and development of primary liver cancer and those potentially eligible for liver transplant is large. Prevalence of NAFLD appears to be increasing, in part due to the increasing numbers of adult and pediatric individuals who are either obese or overweight, have metabolic syndrome or type 2 diabetes, all major risk factors for development of NAFLD [10].

WO 2007/060652 is a previous publication of the present inventor describes compositions comprising beta glycolipids and use thereof for the treatment of immune-related disorder, specifically those that can benefit from modulation of the Th1/Th2 balance toward anti-inflammatory cytokine producing cells. WO 2009/004629, that is also a previous application by the inventor describes methods and compositions involving metadoxine and their use for decreasing and preventing symptoms of alcohol consumption. WO 2009/090656, that is a previous publication by the inventors, describes therapeutic compositions combining beta-glycolipids and antibodies, specifically antibodies targeting CD3, which are beneficial for the treatment of immune-related disorders, also including type 2 diabetes. WO 2009/072132, that is a previous publication of the present inventor, describes methods for treating calcification-related degenerative disorders by means of therapeutic compositions comprising beta glycolipids, specifically applicable to vascular disorders and heart diseases.

Thus, there is a major need for therapeutic compounds, food supplements, food additives, medical foods, botanical drugs and safe drugs that may control blood sugar levels and thereby prevent and ameliorate disorders caused thereby. There is also a need to improve the effect of beta glycolipids and other soy derived products via co-administration of potent adjuvants.

### SUMMARY OF THE INVENTION

In a first aspect, the invention relates to a combined composition for use in at least one of, a method for controlling blood sugar levels in a subject, a method for the treatment of an immune related disorder, a method of treating liver damage and a method for treating, preventing, ameliorating, reducing or delaying the onset of acute or chronic toxic effect of a drug, specifically a drug exhibiting an effect on the liver or on other organs, as well as in a method for enhancing the beneficial effect of a certain drug in a subject. The compositions of the invention comprise as an active ingredient at least one of: (a) at least one natural or synthetic beta-glycolipid or any derivative thereof; (b) at least one polyethoxylated castor oil; (c) at least one adjuvant selected from polyethylene glycol and beta cyclo dextrin or any derivative thereof; and (d) any combination of (a), (b) and (c).

A second aspect of the invention relates to a method for controlling blood sugar levels in a subject, treating an immune related disorder, treating liver damage and for treating, preventing, ameliorating, reducing or delaying the onset of acute or chronic toxic effect of a drug. In more specific embodiments, the methods of the invention comprise providing to a subject a food supplement comprising as an active ingredient at least one of: (a) at least one natural or synthetic beta-glycolipid or any derivative thereof; (b) at least one polyethoxylated castor oil; (c) at least one adjuvant selected from polyethylene glycol and beta cyclo dextrin or any derivative thereof; (d) any combination of (a), (b) and (c); and (e) a composition comprising any one of (a), (b), (c) and (d).

In a further aspect, the invention provides a pharmaceutical composition for use in a method for treating liver damage from any cause in a subject in need thereof. More specifically, such composition may comprise as an active ingredient a therapeutically effective amount of a polyethoxylated castor oil or any derivative or a combination thereof, and optionally further comprising a pharmaceutically acceptable carrier.

Still further, the invention provides a soft or an alcoholic beverage comprising at least one polyethoxylated castor oil or any derivative and at least one a natural or synthetic beta-glycolipid, specifically, GC.

### BRIEF DESCRIPTION OF THE FIGURES

FIG. 1. Beta Glucocerebroside (GC) with Cremophore EL (CE) attenuate increase in serum sugar levels when added to sugar-enriched soft drink, Coca Cola

Figure shows a histogram illustrating AUC (area under the curve) calculated for blood sugar levels in mice receiving orally Coca Cola+GC with CE and mice receiving Coca Cola only (Table 1). Glucose levels were measured at 0, 15, and 30 min after Coca Cola administration. p=0.03 between groups.

FIGS. 2A-2B. CE attenuates increase in serum sugar levels when added to sugar-enriched soft drink, Coca Cola

Figure shows histograms of AUC calculated for blood sugar levels in mice receiving orally Coca Cola+CE and mice receiving Coca Cola only (Table 2). Glucose levels were measured at 0, 15, 30 and 60 min after Coca Cola administration.

FIG. 2A. shows AUC calculated for 30 min, p=0.01 between groups.

FIG. 2B. shows AUC calculated for 60 min p=0.02 between groups.

FIGS. 3A-3B. GC with CE attenuate increase in serum sugar levels when added to sugar-enriched soft drink, Soda Stream Orange flavor

Figure shows histograms of AUC calculated for blood sugar levels in mice receiving orally Soda Stream+CE and mice receiving Soda Stream only (Table 3). Glucose levels were measured at 0, 15, and 30 and 60 min after Soda Stream administration.

FIG. 3A. shows AUC calculated for 30 min, p=0.01 between groups.

FIG. 3B. shows AUC calculated for blood sugar levels after 60 min, p=0.01 between groups.

FIG. 4. CE attenuate increase in serum sugar levels when added to sugar-enriched soft drink, Soda Stream Orange flavor

Figure shows a histogram of AUC calculated for blood sugar levels in mice receiving orally Soda Stream+CE and mice receiving Soda Stream only (Table 4). Glucose levels were measured at 0, 180 min after Soda Stream administration, p=0.04 between groups.

FIGS. 5A-5D. GC with CE exert synergistic effects on serum sugar levels when added to sugar-enriched soft drink, Coke, GTT test

FIG. 5A. shows a graph indicating the blood sugar levels measured at 0, 15, 30, 60, 120, and 180 in mice receiving orally Coca Cola+GC with CE, Coca Cola+GC in Ethanol (EtOH), Coca Cola+CE, Coca Cola+EtOH and mice receiving Coca Cola only, i.e. control (Table 5). Glucose levels were measured by Glucose tolerance test (GTT).

FIG. 5B. shows a histogram of AUC at 30 min GC in ETOH and GC+CE had significant effects, (p=0.012 and p=0.001 compared to control, respectively), while CE alone was not significant.

FIG. 5C. shows a histogram of AUC at 60 min. GC+CE had a significant effect as compared to CE alone or control (p=0.038 and p=0.003, respectively), while CE alone was marginal (p=0.04), suggesting synergism of GC and CE effects.

FIG. 5D. shows a histogram of the total AUC GC+CE had significant effects compared to control or GC in ethanol (p=0.011 and p=0.002)), CE alone was also significant in these conditions (p=0.03).

FIG. 6. GC with CE exert a synergistic protective effect on the immune-mediated liver damage in Concanavalin A (ConA) induced hepatitis

Figure shows a histogram of the alanine aminotransferase (ALT) and aspartate aminotransferase AST levels in ConA mice treated with GC+C:E, GC in ethanol or C:E alone compared to untreated controls (Table 6). GC+CE exerted significant effects in alleviating the immune mediated liver damage (p<0.005).

FIG. 7. GC with CE exert a synergistic protective effect in alleviating the liver damage induced by acetaminophen (APAP)

Figure shows a histogram of ALT levels in mice previously administered with APAP, including mice treated with GC+C:E, GC in ethanol or C:E alone compared to controls (Table 7). GC+CE demonstrated a significant synergistic effect on drug-mediated toxicity to the liver.

FIG. 8. Effect of oral administration of GC+CE with Coca Cola on blood sugar levels in humans

Figure shows the total AUC (75 min.) of sugar levels in human receiving Coca Cola with add-ons of GC in ethanol or GC with CE (Table 8). The data suggests that the phenomenon seen in mice is applicable to humans, as an add-on of GC:CE to Coca Cola had significant and synergistic effects on GTT.

FIG. 9. Effect of oral co-administration of GC alcohol on the alcohol-mediated liver damage

Figure shows ALT levels at 16 hours in mice receiving orally ethanol (EtOH) or EtOH supplemented with GC (Table 9). The results show a significant beneficial effect of oral co-administration of GC and alcohol in alleviating the alcohol-induced liver damage, suggesting that in these conditions GC acts as a liver protector.

FIG. 10. GC with CE when added to sugar-enriched chocolate milk exert a synergistic protective effect on controlling serum sugar levels

Figure shows a histogram of AUC (60 min.) calculated for blood sugar levels in mice receiving orally chocolate milk+GC with CE, chocolate milk with CE alone, or chocolate milk only (controls) (Table 10). Glucose levels were measured at 0, 15, 30 and 60 min after chocolate milk administration. The results demonstrate significant effects of GC or CE and a synergistic effect of GC+CE supplementation on preventing blood sugar increase compared to controls.

FIG. 11. GC with polyethylene glycol or beta cyclo dextrin prevent increase in serum sugar levels when added to sugar-enriched soft drink, Coke

Figure shows a histogram of the total AUC (60 min.) calculated for blood sugar levels in mice receiving orally Coca Cola+GC with polyethylene glycol or beta cyclo dextrin (Table 11). Glucose levels were measured at 0, 15, 30 and 60 min after Coca Cola administration. GC with polyethylene glycol or beta cyclo dextrin add-ons to Coca Cola had beneficial effects on GTT compared with controls.

### DETAILED DESCRIPTION OF THE INVENTION

The present invention is based on surprising and unprecedented findings showing that Cremophor EL (specifically, CE) as well as other adjuvants, such as polyethylene glycol or beta cyclo dextrin and especially in combination with beta-glucosylceramide (GC) have powerful effects. While being aware of protective effects of GC in the reversal of hepatopathologies in hepatitis- and drug-induced liver damage models, the inventors presently found that GC with CE act synergistically as liver protectors, and moreover as modulators and controllers of blood sugar levels. This finding is moreover unanticipated in view of the longstanding experience with CE as presumably neutral excipient and emulsifying agent. Validity of this finding was further substantiated by the fact that CE alone was capable of, albeit to a lesser extent, inducing the same effects.

These findings have potential applicability on several levels. First, they serve basis for the development of new therapeutic compounds for the treatment of hepatopathologies in a large number of clinical contexts, including alleviation of immuno-mediated liver damage, drug-induced liver damage, alcohol-induced liver damage, as well as cirrhosis and/or hepatic failure ensuing from infections, cancer, alcoholic steatohepatitis, non-alcoholic steatohepatitis (NASH or NAFLD) and other chronic liver diseases.

Second, based on finding of GC and CE effects on blood sugar (or glucose) levels, as well as the effects of combinations of GC with other adjuvants such as polyethylene glycol or beta cyclo dextrin, it is conceivable that the afore-mentioned therapeutic compounds could be effective against other clinical conditions in which disruption of blood glucose homeostasis plays an important role, such as diabetes types 1 and 2, gestational diabetes, pre-diabetes, autoimmune diabetes and any state of altered insulin resistance.

Third, but not less important, these findings lead to notion that natural or synthetic GC and CE in combination or alone, or combinations of GC with other adjuvants such as polyethylene glycol or beta cyclo dextrin, may be used as “bouncers” in preventing the development of pre-clinical conditions ensuing from exposure to exceeding increased or decreased blood sugar levels, as occurs after consumption of sugar-enriched foods and beverages or alcohol, respectively. In this context, GC and/or CE or combinations of GC with other adjuvants such as polyethylene glycol or beta cyclo dextrin rather than being applicable to patients as therapeutic agents, are in fact food additives meant to normalize risks ensuing from modern lifestyle and standard of living to which are subjected normal individuals. Said therapeutic compounds, drugs, medical foods, food supplements and food additive, especially in form of add on to sugar sweetened and/or alcoholic beverages are especially beneficial for preventing common conditions, such as weight gain, alcohol intoxication and also more severe presentations, such as obesity and alcohol withdrawal syndrome.

Thus, in a first aspect, the invention relates to a combined composition for use in at least one of, a method for controlling blood sugar levels in a subject, a method for the treatment of an immune related disorder, a method of treating liver damage and a method for treating, preventing, ameliorating, reducing or delaying the onset of acute or chronic toxic effect of a drug, as well as in a method for enhancing the ameliorating and beneficial effect of a certain drug in a subject. The composition of the invention comprising as an active ingredient at least one of: (a) at least one natural or synthetic beta-glycolipid or any derivatives thereof; (b) at least one polyethoxylated castor oil or any derivative thereof; (c) at least one adjuvant such as polyethylene glycol or beta cyclo dextrin or any derivative thereof; and (d) any combination of (a), (b) and (c). It should be appreciated that in some embodiments, the compositions of the invention may optionally further comprise at least one of carrier/s, diluent/s, excipient/s or any pharmaceutically acceptable version thereof.

In some embodiments, the natural or synthetic beta-glycolipid comprised in the composition of the invention may be any one of a glycosphingolipid, glucosylceramide, monosaccharide ceramide, galatosylceremide, lactosylceramide, gal-gal-glucosyl-ceramide, GM2 ganglioside, GM3 ganglioside, globoside or any soy derivative or a combination thereof.

The compositions as well as the methods of the present invention described herein later, comprise as an active ingredient, and therefore relate to a group of glycolipids (i.e. lipids with a carbohydrate moiety) designated as β-glycolipids. The β-glycolipid of the invention may be any synthetic or natural β-glycolipid or any derivative or combination thereof. Further, the β-glycolipid of the invention may be selected from the group of glycosphingolipids, of a natural or non-natural source, with any number of carbons and double bonds and with any length of the lipid tail of the molecule. More specifically, the β-glycolipid of the invention may be a glucosylceramide, a monosaccharide ceramide, a galatosylceremide, a lactosyl-ceramide, a gal-gal-glucosyl-ceramide, GM2 ganglioside, GM3 ganglioside, or globoside, or any soy derived product which have been specifically associated with an immunomodulatory effect.

In certain preferred embodiments of the invention, the above β-glycolipid is a natural or synthetic β-glucosylceramide (GC). The natural GC is the only glycosphingolipid common to plants, fungi and animals, in all of which it constitutes a major component of the outer layer of the plasma membrane. GC is considered to be the principal glycosphingolipid in plants. In animals, GC is a major constituent of skin lipids, where it is essential for lamellar body formation in the stratum corneum and to maintain the water permeability barrier of the skin. Lower levels of GC are found in cells of the spleen, erythrocytes, and nervous tissues, especially the neurons.

In certain embodiments, the combined composition of the invention may comprise any soy derivative. Under soybean is meant seeds or beans of a plant belonging to the genus Glycine, including the two subgenera, Glycine and Soja. Further pertinent thereto, genetically modified soybeans which may include, among others, glyphosate-tolerant or herbicide-tolerant soybeans which constitute now the majority (93%) of the commercial market in the US.

Apart from extracts derived for the soybean, other extracts may be derived from the solvent extraction of soy pollens into oil which contains tri- and di-glicerydes, free fatty acids and phosphatides, as well as extracts derived from aqueous-ethanol extraction left after the solvent extraction, which contains soy protein, isoflavones, sugars (oligo-, di-, mono-), and lipids (including phosphatides, phytosterols, saponins).

In certain embodiments, the combined composition of the invention may comprise any soy derivative. As noted above, soy derivative may comprise any soy preparation or extract. With respect to the at least one soybean extract, it is appreciated that, according to some embodiments of the combined compositions and methods of the invention, such extract may be an enzymatic soybean extract, a hexane extract or aqueous extract.

More specifically, the term “extract” refers to any substances obtained by extracting soy beans using either enzymatic extracts, organic solvents or by hydrophilic solvents. More specifically, the term “extract” refers to any substances obtained by extracting soy beans using either organic solvents such as, for example, hexane, ethyl-acetate or isopropyl-alcohol, or by hydrophilic solvents, such as water. The extracts may be dried after said extraction and may be further extracts by any extraction method, independently from previous extraction steps. Such steps may be repeated independently. Furthermore, other extraction techniques may be employed, non-limiting examples of which include chromatography, including size-exclusion, hydrophobic interaction, and anion and cation exchangers, differential centrifugation, differential precipitation (for example, using ammonium sulfate), differential filtration and dialysis.

Many extraction methods may be used for producing the soybean extracts of the invention.

For example, at least one of an aliphatic organic solvent and water, or supercritical carbon dioxide gas may be used as an extractant for extraction of phospholipids from the soybean, preferably a defatted soybean material. The aliphatic organic solvent is preferably a saturated hydrocarbon, an alcohol, a mixed solvent of saturated hydrocarbon and alcohol, or a mixed solvent of halogenated hydrocarbon and alcohol. It is preferable that the extract be at least one of hexane, ethanol, methanol, hydrous ethanol, isopropyl alcohol, acetonitrile and acetone.

The extract may be enriched with aromatic chromophore containing compounds including the isoflavones genistein, daidzein, formononetin and biochanin and/or their glycosides, and for administration it is generally provided in association with one or more pharmaceutically acceptable carriers, excipients, auxiliaries, and/or diluents.

Other procedures for specifically enriching or removing soybean isoflavones include differential extraction with organic solvents, based on the differing solubility of aromatic chromophore containing compounds in certain organic solvents.

As described in the art, extraction of soybeans may also incorporate enzymatic treatment of said soybeans, whether before, during or after mechanical disruption and/or chemical extraction of said soybeans. Therefore, enzymatic treatment of the plant material is specifically contemplated herein. Enzymes used for said extraction include cellulase, hemicellulase, pectinase, protease and other carbohydrases. The use of enzymatic treatment may be carried out under various moisture and temperature conditions suitable for optimal enzyme activity as known in the art. When performing enzymatic treatment of the plant material during chemical extraction, it is appreciated that the solvent and conditions used must be compatible with the maintenance of adequate enzymatic activity, and care must be taken not to inhibit the enzyme activity or to denature it.

According to more specific embodiments, the glucosylceramide comprised in the composition of the invention may be a beta glucosylceramide (GC).

Further, the compositions and methods of the present invention relate to castor oil and specifically to synthetic castor oil derivatives. Castor oil as meant herein relates to a natural vegetable oil obtained from seeds of the castor oil plant (Ricinus communis). The FDA has categorized castor oil as “generally recognized as safe and effective” (GRAS) for over-the-counter use as a laxative. Castor oil or synthetic castor oil derivatives such as polyethoxylated castor oil have been approved for human use as vehicles for oral and intravenous administration of water-insoluble therapeutic compounds. In naturopathy, castor oil has been promoted as a treatment for a variety of human health conditions.

The term ‘Ethoxylated Castor Oil’ (also Polyoxyl Castor Oil, Polyoxyl Castor Oil, Polyethylene Glycol Castor Oil, Castor Oil Ethoxylates and Polyethoxylated Castor Oil) refers to a nonionic surfactant having many industrial applications. Polyoxyethylene castor oil derivatives are complex mixtures of various hydrophobic and hydrophilic components. In the polyethoxylated castor oil, the hydrophobic constituents comprise about 80% of the total mixture, the main component being glycerol polyethylene glycol ricinoleate. Other hydrophobic constituents include fatty acid esters of polyethylene glycol along with some unchanged castor oil. The hydrophilic part consists of polyethylene glycols and glycerol ethoxylates.

Further, ethoxylated castor oil is also referred to as a mixture of triricinoleate esters of ethoxylated glycerol with small amounts of polyethyleneglycol (macrogol) ricinoleate and the corresponding free glycols. Polyoxyethylene castor oil derivatives are nonionic surfactants used in oral, topical and parenteral pharmaceutical formulations.

In yet some further embodiments, the derivative of polyethoxylated castor oil of the composition of the invention may be Cremophore EL. As such, the present invention specifically relates to a version of polyethoxylated castor oil known as Cremophor EL or more recently Kolliphor EL (registered trademark of BASF Corp) and also polyoxyethylenglyceroltriricinoleat 35 (DAC), polyoxyl 35 castor oil (USP/NF), obtained by reacting ethylene oxide with castor oil (molar ratio 35:1). The main component of Cremophor EL is glycerol-polyethylene glycol ricinoleate, which, together with fatty acid esters of polyethylene glycol, represents the hydrophobic part of the product. The smaller, hydrophilic part consists of polyethylene glycols and ethoxylated glycerol. Due to this particular composition, Cremophor EL is capable to stabilize emulsions of nonpolar materials in aqueous solutions, thus making it a universal nonionic emulsifying agent for the pharmaceutical, cosmetic and food industries. Some anti-neoplastic agents (e.g. Taxol, Taxotere) were formulated in Cremophor EL and ethanol to enhance drug solubility and therapeutic effect. When describing the present invention, the terms emulsifying agents, excipient and surfactant are interchangeable.

Specifically, Cremophor EL (CAS Registry number 63393-92-0) (Synonyms Macrogolglycerol ricinoleate, PEG-35 castor oil, Polyoxyl 35 hydrogenated castor oil, Polyoxyl-35 castor oil) denotes a derivative of castor oil or an ester with ethoxylated glycerol of Molecular Formula C₅H₁₂O₄; Molecular Weight:136.14638 [g/mol]; Formal Charge:0; Boiling Point 290° C. at 760 mmHg; Flash Point 160° C.

Further, the term CE or C:E designates preparation of Cremophor EL in ethanol (1:1 v/v) and it represents 30% v/v when emulsified in PBS. According to some specific embodiments, the Cremophore EL may be dissolved in or combined with EtOH. More specifically, the C and the E (EtOH) ratio may range between about 1:0 to 1:999999, more specifically, 1:1 to 1:99999, 1:1 to 1:9999, 1:1 to 1:999, 1:1 to 1:99, 1:1 to 1:9.

Nevertheless, it should be appreciated that the Cremophor of the invention may be prepared or dissolved in any other solvent.

As noted above, the combined compositions of the invention comprise at least two active agents, specifically, GC and C:E. It should be appreciated that any quantitative ratio of the combined compounds may be used. As a non-limiting example, a quantitative ratio used between any of the compounds may be: 1:1, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100, 1:200, 1:300, 1:400, 1500, 1:750, 1:1000. It should be further noted that where the combination of the invention comprises more than two compounds, specifically, where additional therapeutic agents are added, the quantitative ratio used may be for example, 1:1:1, 1:2:3, 1:10:100, 1:10:100:1000 etc.

In certain embodiments, the combined composition of the invention may comprise an adjuvant such as any one of polyethylene glycol or beta cyclo dextrin or any derivative thereof. The term “adjuvant” as used herein refers to a pharmacological agent that modifies and enhances the effect of other active agents. It should be noted that the specific adjuvants indicated herein were now surprisingly shown by the invention as exerting a therapeutic effect/s as active main ingredients and not only as additional enhancing or inherent agents.

In some specific embodiments, the combined compositions of the invention may comprise GC and Polyethylene glycol or any derivatives thereof. Polyethylene glycol (PEG) is a polyether compound PEG is also known as polyethylene oxide (PEO) or polyoxyethylene (POE), depending on its molecular weight. PEG, PEO, or POE refers to an oligomer or polymer of ethylene oxide. The three names are chemically synonymous, PEG refer to oligomers and polymers with a molecular mass below 20,000 g/mol, PEO to polymers with a molecular mass above 20,000 g/mol, and POE to a polymer of any molecular mass. PEG and PEO are liquids or low-melting solids, depending on their molecular weights. PEGs are prepared by polymerization of ethylene oxide and are commercially available over a wide range of molecular weights from 300 g/mol to 10,000,000 g/mol. While PEG and PEO with different molecular weights find use in different applications, and have different physical properties (e.g. viscosity) due to chain length effects, their chemical properties are nearly identical. Different forms of PEG are also available, depending on the initiator used for the polymerization process—the most common initiator is a monofunctional methyl ether PEG, or methoxypoly(ethylene glycol), abbreviated mPEG. Lower-molecular-weight PEGs are also available as purer oligomers, referred to as monodisperse, uniform, or discrete.

PEG is soluble in water, methanol, ethanol, acetonitrile, benzene, and dichloromethane, and is insoluble in diethyl ether and hexane. It is coupled to hydrophobic molecules to produce nonionic surfactants. When attached to various protein medications, polyethylene glycol allows a slowed clearance of the carried protein from the blood. This makes for a longer-acting medicinal effect and reduces toxicity, and allows longer dosing intervals.

PEG is used as an excipient in many pharmaceutical products. Lower-molecular-weight variants are used as solvents in oral liquids and soft capsules, whereas solid variants are used as ointment bases, tablet binders, film coatings, and lubricants.

In more specific embodiments, The term ‘Polyethylene Glycol’ (CAS Registry number 25322-68-3; CA Index Name: Poly(oxy-1,2-ethanediyl), α-hydro-ω-hydroxy-) denotes an addition polymer of ethylene oxide and water, represented by the formula H(OCH2CH2)nOH, denoted herein as Formula I:

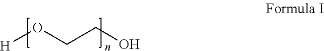

in which n represents the average number of oxyethylene groups. In some embodiments, the average molecular weight is not less than 95.0% and not more than 105.0% of the labeled nominal value if the labeled nominal value is below 1000; it is not less than 90.0% and not more than 110.0% of the labeled nominal value if the labeled nominal value is between 1000 and 7000; it is not less than 87.5% and not more than 112.5% of the labeled nominal value if the labeled nominal value is above 7000. It may contain a suitable antioxidant.

Surprisingly, the invention now shows the effect of PEG in lowering blood sugar levels (FIG. 11). Thus, in certain embodiments, the invention provides a combination of GC and PEG for use as described by the invention.

The term PEG designates preparation of PEG in ethanol (1:1 v/v) and it represents 30% v/v when emulsified in PBS. According to some specific embodiments, the PEG may be dissolved in or combined with EtOH. More specifically, the PEG and the E (EtOH) ratio may range between about 1:0 to 1:999999, more specifically, 1:1 to 1:99999, 1:1 to 1:9999, 1:1 to 1:999, 1:1 to 1:99, 1:1 to 1:9. The PEG of the invention may be prepared or dissolved in any other solvent. As noted above, in some alternative embodiments, the combined compositions of the invention may comprise at least two active agents, specifically, GC and PEG. It should be appreciated that any quantitative ratio of the combined compounds may be used. As a non-limiting example, a quantitative ratio used between any of the compounds may be: 1:1, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100, 1:200, 1:300, 1:400, 1500, 1:750, 1:1000. It should be further noted that where the combination of the invention comprises more than two compounds, specifically, where additional therapeutic agents are added, the quantitative ratio used may be for example, 1:1:1, 1:2:3, 1:10:100, 1:10:100:1000 etc.

Still further alternative embodiments of the invention encompass the provision of combined compositions comprising as active ingredients GC and Beta cyclo dextrin (BCD).

Cyclodextrins (sometimes called cycloamyloses) are a family of compounds made up of sugar molecules bound together in a ring (cyclic oligosaccharides). Cyclodextrins are produced from starch by means of enzymatic conversion. They are used in food, pharmaceutical, drug delivery, and chemical industries, as well as agriculture and environmental engineering. Cyclodextrins are composed of 5 or more α-D-glucopyranoside units linked 1->4, as in amylose (a fragment of starch). The 5-membered macrocycle is not ural. Recently, the largest well-characterized cyclodextrin contains 32 1, 4-anhydroglucopyranoside units, while as a poorly characterized mixture, at least 150-membered cyclic oligosaccharides are also known. Typical cyclodextrins contain a number of glucose monomers ranging from six to eight units in a ring, creating a cone shape: α (alpha)-cyclodextrin: 6-membered sugar ring molecule; β (beta)-cyclodextrin: 7-membered sugar ring molecule; γ (gamma)-cyclodextrin: 8-membered sugar ring molecule. α- and γ-cyclodextrin are being used in the food industry. As α-cyclodextrin is a soluble dietary fiber, it can be found as Alpha Cyclodextrin (soluble fiber) on the list of ingredients of commercial products.

Because cyclodextrins are hydrophobic inside and hydrophilic outside, they can form complexes with hydrophobic compounds. Thus they can enhance the solubility and bioavailability of such compounds. This is of high interest for pharmaceutical as well as dietary supplement applications in which hydrophobic compounds shall be delivered. Alpha-, beta-, and gamma-cyclodextrin are all generally recognized as safe by the FDA. In the food industry, cyclodextrins are employed for the preparation of cholesterol free products.

More specifically, the term ‘β-Cyclodextrin’ (CAS Registry number 7585-39-9; Synonyms Cycloheptaamylose, Cyclomaltoheptaose, β-cycloamylose, cycloheptaglucan, cycloheptaglucosan, Betadex) denotes a cyclodextrin composed of seven α-(1-4) linked D-glucopyranose units C₄₂H₇₀O₃₅; Molecular Weight 1134.98 [g/mol]

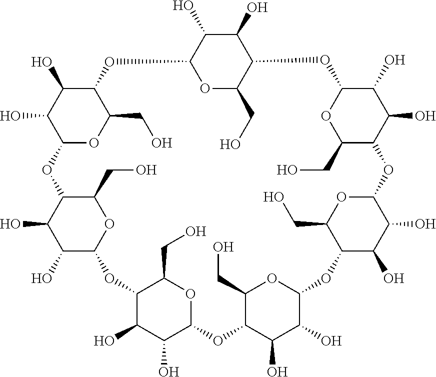

In more specific embodiments, the invention provides combined compositions comprising as active ingredients GC and Methyl-β-cyclodextrin.

Both β-cyclodextrin and methyl-β-cyclodextrin (MβCD) remove cholesterol from cultured cells. The methylated form MβCD was found to be more efficient than β-cyclodextrin. The water-soluble MβCD is known to form soluble inclusion complexes with cholesterol, thereby enhancing its solubility in aqueous solution. MβCD is employed for the preparation of cholesterol-free products: the bulky and hydrophobic cholesterol molecule is easily lodged inside cyclodextrin rings that are then removed. MβCD is also employed in research to disrupt lipid rafts by removing cholesterol from membranes.

Further, the term BCD designates preparation of BCD in ethanol (1:1 v/v) and it represents 30% v/v when emulsified in PBS. According to some specific embodiments, the BCD may be dissolved in or combined with EtOH. More specifically, the BCD and the E (EtOH) ratio may range between about 1:0 to 1:999999, more specifically, 1:1 to 1:99999, 1:1 to 1:9999, 1:1 to 1:999, 1:1 to 1:99, 1:1 to 1:9.

Nevertheless, it should be appreciated that the BCD of the invention may be prepared or dissolved in any other solvent.

As noted above, the combined compositions of the invention comprise at least two active agents, specifically, GC and BCD. It should be appreciated that any quantitative ratio of the combined compounds may be used. As a non-limiting example, a quantitative ratio used between any of the compounds may be: 1:1, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100, 1:200, 1:300, 1:400, 1500, 1:750, 1:1000. It should be further noted that where the combination of the invention comprises more than two compounds, specifically, where additional therapeutic agents are added, the quantitative ratio used may be for example, 1:1:1, 1:2:3, 1:10:100, 1:10:100:1000 etc.

Yet in other specific embodiments, the invention provides combined compositions comprising as active ingredients GC and at least one other inactive ingredient that in the present case may work as an active ingredient and synergistically lower blood sugar levels. The term ‘inactive ingredient’, as defined by the US Food and Drug Administration (FDA), refers to an excipient, solvent, binding material or preservative that is generally considered inert or pharmacologically inactive. There are examples however, such as in the case of the present invention, that inactive ingredient in certain combinations and conditions become active ingredients (for examples see further below), this term also encompasses an agent that combines to an active ingredient to facilitate drug transport.

Examples of FDA approved inactive ingredients which are used in drug and food industry and therefore may be applicable to compositions of the present invention include, although not limited to the following ingredients indicated herein after. Thus, in specific embodiments, the present invention may provide a combined composition comprising at least one natural or synthetic beta-glycolipid, specifically, GC and at least one of: acacia, acetic acid, vitamins such as alpha-tocopherol (vitamin E1), ascorbic acid (vitamin C), ascorbyl palmitate (i.e. a fat-soluble form of vitamin C recognized as GRAS), riboflavin (vitamin B2), certain amino acids such as glycine, phenylalanine, and variants thereof such as cysteine hydrochloride, certain salts in particular certain aluminum salts (e.g. aluminum stearate), and sodium, magnesium, potassium and calcium salts (e.g. sodium alginate), benzalkonium chloride, betadex (i.e. beta-cyclodextrin referred to above), butylparaben, Candelilla wax, Carrageenan, castor oil (or a derivative thereof as mentioned above) and other vegetative oils such as coconut, corn, cottonseed, palm kernel, sesame, sunflower, olive, peanut and soybean oils, including hydrogenated variants thereof, cellulose compounds (also microcrystalline cellulose), cetostearyl alcohol including cetyl alcohol, cetylpyridium chloride, citric acid, croscarmellose sodium (also sodium CMC recognized as GRAS), diethyl phthalate (DEP), dimethylaminoethyl methacrylate-butyl methacrylate-methyl methacrylate copolymer (brand name Eudragit EPO) and other Eudragit derivatives, docusate sodium (also dioctyl sodium sulfosuccinate), lanolin, lecithin, ethyl cellulose, fumaric acid, gelatin, glucosamine, glycerin (also glycerol, glycerine), glyceryl behenate, glyceryl distearate, glyceryl monostearate, glyceryl triacetate, isobutylparaben, medium chain triglycerides (MCT), methylparaben, propylparaben and butylparaben, monosodium citrate (recognized as GRAS), poloxamers of common available grades 68, 88, 98, 108, 124, 188, 237, 338, and 407, polycarbophil, polyethylene glycol (PEG referred to above of average molecular weight in the range of 300-8000), polygalacturonic acid, polyplasdones, polysorbates, polyvinylpyrrolidone, povidones, propyl gallate, propylparaben, sorbic acid, succinic acid, tartaric acid, taurine, tragacanth, triacetin and triethyl citrate (recognized as GRAS).

Further inactive ingredients such as sulfites, benzoates (i.e. parabens), benzoic acid, boric acid, bronopololeic acid, butylated hydroxyanisole (BHA, E320), butylated hydroxytoluene (BHT, E321), chlorobutanol, chlorocresol, dimethyl sulfoxide, sorbitan and sorbitan derivatives may be used in certain proportions for the same purpose, as these agents have been reported to cause adverse reactions in some patients. Thus, in some specific embodiments, the invention provide combined compositions comprising an effective amount of at least one natural or synthetic beta-glycolipid, specifically, GC and at least one of: sulfites, benzoates (i.e. parabens), benzoic acid, boric acid, bronopololeic acid, butylated hydroxyanisole (BHA, E320), butylated hydroxytoluene (BHT, E321), chlorobutanol, chlorocresol, dimethyl sulfoxide, sorbitan and sorbitan derivatives. It must be appreciated that the combined compositions defined above, as well as any combined composition defined and provided by the invention may be used as add-on to any a solid, semi-solid or liquid food, beverage, food additive, food supplement, medical food, botanical drug, drug and/or any type of pharmaceutical compound.

Still further, the invention further encompasses any soft or an alcoholic beverage comprising at least one natural or synthetic beta-glycolipid, specifically, GC and at least one of any of the ingredients indicated herein above.

Still further, the invention provides methods as specified by the invention using any of the combined compositions described above or any combinations thereof. In certain embodiments the composition of the invention may be formulated in a formulation adapted for add-on to a solid, semi-solid or liquid food, beverage, food additive, food supplement, medical food, botanical drug, drug and/or any type of pharmaceutical compound.

In some embodiments, the add-on composition according to the invention may be formulated as a food additive, food supplement or medical food. In other embodiment, such add-on composition of the invention may be further added or combined with botanical drugs, drugs or any type of pharmaceutical products. The term ‘add-on’ as used herein is meant a composition or compound that may be added to existing compound, composition or material enhancing desired properties thereof or alternatively, adding specific desired property to an existing compound.

More specifically, in certain embodiments, the combined composition of the invention may be an add-on to a food supplement, or alternatively, may be used as a food supplement. A food supplement, the term coined by the European Commission for Food and Feed Safety, or a dietary supplement, an analogous term adopted by the FDA, relates to any kind of substances, natural or synthetic, with a nutritional or physiological effect whose purpose is to supplement the normal diet. In this sense, this term also encompasses food additives and dietary ingredients. Further, under the Dietary Supplement Health and Education Act of 1994 (DSHEA), a statute of US Federal legislation, the term dietary supplement is defined as a product (other than tobacco) intended to supplement the diet that bears or contains one or more of the following dietary ingredients: a vitamin, a mineral, an herb or other botanical, an amino acid, a dietary substance for use by man to supplement the diet by increasing the total dietary intake, or a concentrate, metabolite, constituent, extract, or combination of any of the aforementioned ingredients.

Food or dietary supplements are marketed a form of pills, capsules, powders, drinks, and energy bars and other dose forms. Unlike drugs, however, they are mainly unregulated, i.e. marketed without proof of effectiveness or safety. Therefore, the European and the US laws regulate dietary supplements under a different set of regulations than those covering “conventional” foods and drug products. According thereto, a dietary supplement must be labeled as such and be intended for ingestion and must not be represented for use as conventional food or as a sole item of a meal or a diet.

In yet some further embodiments, the combined composition of the invention may be an add-on to medical foods. Further in this connection should be mentioned medical foods, which are foods that are specially formulated and intended for the dietary management of a disease that has distinctive nutritional needs that cannot be met by normal diet alone. The term medical food, as defined in the FDA's 1988 Orphan Drug Act Amendments is a food which is formulated to be consumed or administered enterally under the supervision of a physician and which is intended for the specific dietary management of a disease or condition for which distinctive nutritional requirements, based on recognized scientific principles, are established by medical evaluation. Hence, medical foods are subject to the general food and safety labeling requirements of the Federal Food, Drug, and Cosmetic Act. Medical foods are usually classified as nutritionally complete or incomplete formulas, formulas for metabolic disorders and oral rehydration products. Notable examples of the above include gamma-linolenic acid (GLA) and/or a short chain omega-6 fatty acid sourced from the seeds of the borage plant for management of allergic conditions; slowly digested carbohydrates for maintenance of optimal blood sugar levels especially in patients with diabetes; and glutamine for nourishment of the gastrointestinal (GI tract) in metabolically stressed patients.

Also pertinent to the present context are botanical drugs. Thus, in further embodiments, the composition of the invention may be an add-on to a botanical drug. As used herein botanical drug are products that are intended for use in the diagnosis, cure, mitigation, treatment or prevention of disease in humans. A botanical drug product consists of vegetable materials, which may include plant materials, algae, macroscopic fungi, or combinations thereof. A botanical drug product may be available as (but not limited to) a solution (e.g., tea), powder, tablet, capsule, elixir, topical, or injection. Botanical drug products often have unique features, for example, complex mixtures, lack of a distinct active ingredient, and substantial prior human use. Fermentation products and highly purified or chemically modified botanical substances are not considered botanical drug products. According to the FDA Guidance for Industry, a botanical product may be a food (including a dietary supplement), a drug (including a biological drug), a medical device (e.g., gutta-percha), or a cosmetic. Further, botanical drugs may include botanical ingredients in combination with either a synthetic or highly purified drug or a biotechnology derived or other naturally derived drug. In the same way, botanical drugs may also contain animals or animal parts (e.g., insects, annelids, shark cartilage) and/or minerals or a combination thereof.

Also pertinent to the present context are any type of drugs or therapeutic compounds. Thus, in further embodiments, the composition of the invention may be an add-on to any type of drugs or therapeutic compounds administered orally, intravenously, intradermaly, by inhalation or intrarectaly.

In certain embodiments, the combined composition of the invention may comprise any type of gut hormone. In more specific embodiments, the combined composition of the invention may comprise as an additional therapeutic agent at least one gut hormone. In some alternative embodiments, the combined composition of the invention may be used as an add-on product for concomitant administration of at least one gut hormone. In more particular but non-limiting embodiments, gut hormone include Ghrelin, Cholecystokinin, Cholecystokinin, Peptide YY, Pancreatic polypeptide, Amylin, Glucose-dependent insulinotropic polypeptide, Glucagon-like peptide-1, Glucagon-like peptide-2 and Oxyntomodulin. In more specific embodiments the combined composition of the invention may comprise Ghrelin. As used herein Ghrelin is a peptide hormone released from the stomach and liver and is often referred to as the “hunger hormone” since high levels of it can be found in individuals that are fasting. Ghrelin antagonistic treatments can be used to treat illnesses such as anorexia and loss of appetites in cancer patients. Ghrelin treatments for obesity are still under intense scrutiny and no conclusive evidence has been reached. This hormone stimulates growth hormone release. In yet some further embodiments the combined composition of the invention may further comprise Cholecystokinin. As used herein Cholecystokinin is responsible for gall bladder secretions, gastrointestinal motility as well as pancreatic exocrine secretions. Peptide YY that may be also comprised within the composition of the invention is involved mostly in satiation modulation. Still further, the combined compositions of the invention may comprise pancreatic polypeptide. Pancreatic polypeptide function is most apparent in control of gastrointestinal motility and satiation. In further embodiments Amylin may be also added to the combined compositions of the invention. Amylin controls glucose homeostasis and gastric motility. Further embodiments relate to the addition of Glucose-dependent insulinotropic polypeptide to the combined compositions of the invention. Glucose-dependent insulinotropic polypeptide possesses an acute influence on food intake through its effects on adipocytes. In further embodiments, Glucagon-like peptide-1 may be added to the compositions of the invention. Glucagon-like peptide-1 has an effect on incretin activity as well as satiation. In other embodiments, Glucagon-like peptide-2 may be added to the compositions of the invention. Glucagon-like peptide-2 is responsible for gastrointestinal motility and growth. Further embodiments relate to the addition of Oxyntomodulin to the combined compositions of the invention. Oxyntomodulin plays a role in controlling acid secretion and satiation. Each of these can be added to glycopilids or to CE or to be added by itself or in combination with other compounds.

In some embodiments, the composition of the invention may be adapted for add-on a food and/or beverage that comprise an increased content of sugar and/or alcohol or to a food or beverage that may be associated with increase in blood sugar or alcohol level via alteration of the insulin resistance state or the capability to alter alcohol metabolism by the body.

As previously mentioned, temporary fluctuations of blood glucose levels may develop under various conditions, among which consumption of sugar sweetened or alcoholic beverages represent a significant contributing factor.

In this context, a sugar sweetened beverage is any beverage with added sugar, including for example fruit or fruit-flavored drinks, flavored water or sodas, energy drinks, coffees, teas, chocolate milk and nonalcoholic wines and beers. For the purpose of describing the invention, the terms added sugar, sugar sweetened and high sugar content are interchangeable. Risks of weight gain, obesity and diabetes which have been linked to consumption of sweetened beverages will be discussed further below.

An alcoholic beverage is a drink typically containing 0.1-95% alcohol, most commonly ethanol but occasionally other alcohols too. Alcoholic beverages include beers, wines, and spirits (distilled beverages). For the purpose of the present invention, the term an alcoholic beverage encompasses any kind of alcohol containing beverage produced by process of fermentation or distillation or both, or any type of food or drink that directly or indirectly affect the metabolism of alcohol. In the same way will be discussed consequences of alcohol consumption, such as alcohol intoxication, hangover and liver damage, well as the link between alcohol consumption and blood sugar levels.

In some embodiments, the composition of the invention is applicable for controlling blood sugar levels in a subject. More specifically, such control may be inhibiting increase or decrease in blood sugar levels or alternatively, altering the insulin resistance state in the treated subject.

As meant herein, the terms blood sugar level or blood glucose level imply molar concentration of glucose present in the blood of an organism (human or animal). Glucose, being with some exceptions the primary source of energy for all body's cells, is transported from the intestines or liver to body cells via the bloodstream and is made available for cell absorption via the hormone insulin, produced primarily in the pancreas. The body's homeostatic mechanism keeps blood glucose levels within a narrow range by means of several interacting systems, of which hormone regulation is the most important. There are two types of mutually antagonistic metabolic hormones affecting blood glucose levels:
catabolic hormones (such as glucagon, cortisol and catecholamines) which increase blood glucose;
and one anabolic hormone (insulin) which decreases blood glucose.

Glucose levels are usually lowest in the morning, before the first meal of the day (termed “the fasting level”) and rise after meals for an hour or two by a few millimolar. Blood sugar levels outside the normal range may be an indicator of a medical condition. A persistently high level is referred to as hyperglycemia; low levels are referred to as hypoglycemia. Diabetes mellitus is characterized by persistent hyperglycemia from any of several causes, and is the most prominent disease related to failure of blood sugar regulation. Intake of alcohol causes an initial surge in blood sugar, and later tends to cause levels to fall. Certain drugs can also increase or decrease glucose levels.

Blood glucose levels are expressed is in terms of a molar concentration measured in mmol/L (millimoles per litre; or millimolar, abbreviated mM); in the US, blood glucose is measured as mass concentration in mg/dL (milligrams per decilitre). Since the molecular weight of glucose C₆H₁₂O₆ is 180, the difference between the two scales is a factor of 18, so that 1 mmol/L of glucose is equivalent to 18 mg/dL.

One of the important features of the present invention is preventing or reducing temporary fluctuations of blood glucose levels resulting from consumption of sugar-enriched foods and beverages, thus enabling to keep the blood glucose levels within the normal or recommended range.

Under the terms normal or recommended blood glucose levels is meant, in humans, for non-diabetics the mean normal levels (tested while fasting) should be between 70 to 100 mg/dL (3.9 to 5.5 mmol/L). A body's homeostatic mechanism, when operating normally, restores the blood sugar level to the normal range. According to the American Diabetes Association, blood sugar levels for those without diabetes and who are not fasting should be below 125 mg/dL. The blood glucose target range for diabetics should be 90-130 mg/dL before meals and less than 180 mg/dL after meals.

According to other estimates, the normal blood glucose level in humans in fasting is approximately 4 mmol/L (4 mM or 72 mg/dL); shortly after a meal the blood glucose level may rise temporarily up to 7.8 mM (140 mg/dL); when operating normally the body restores blood sugar levels to a range of 4.4 to 6.1 mM (82 to 110 mg/dL). And for people with type 1 or type 2 diabetes blood sugar level targets are: before meals—4 to 7 mM for; after meals—under 9 mM for people with type 1 and 8.5 mM for people with type 2; children with type 1 diabetes have a greater upper limit for their blood sugar levels by 1 mM.

In this connection, it should be also understood under blood glucose levels is meant arterial, venous and capillary blood glucose levels, which may be comparable or distinct, when fasting or after meals.

Further, the present invention may be applicable in conjunction with measuring or monitoring blood glucose levels by means of any manufactured technology enabling direct to customer glucose blood testing, e.g. a disposable test-strip or an electronically-base device. This is particularly applicable for subjects with diabetes or insulin resistance.

Further, the terms preventing, reducing, attenuating, minimizing, inhibiting or controlling fluctuations of blood glucose levels are meant to convey preventing, reducing or controlling increase as well as decrease in blood sugar levels, i.e. increase or decrease of at least about 0.1% 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60% or more.

In more specific embodiments, the compositions of the invention may attenuate, decrease, inhibit, prevent, reduce or minimize the increase or elevation in blood sugar levels caused by high sugar beverages or foods in at least about 0.1% 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60% or more, specifically, 65%, 70%, 75%, 80%, 85%, 90%, 95% or more.

As previously mentioned, temporary fluctuations of blood glucose levels may develop under various conditions, among which consumption of sugar sweetened or alcoholic beverages represent a significant contributing factor. In addition, alteration of blood sugar levels can occur following the use of compounds or in other states where the level of insulin resistance is changed.

In fact, alcohol interferes with all three sources of glucose and the hormones needed to maintain healthy blood glucose levels. The greatest impact is seen in those who drink heavily on a frequent basis. Heavy drinkers deplete their glycogen stores within a few hours when their diet does not provide a sufficient amount of carbohydrates. Over time, excessive alcohol consumption can decrease insulin's effectiveness, resulting in high blood sugar levels; according to certain estimates 45% to 70% of people with alcoholic liver disease had either glucose intolerance or diabetes.

Alcohol can also negatively impact blood sugar levels each time that it is consumed, regardless of the frequency of consumption. Research has shown that acute consumption increases insulin secretion, causing low blood sugar (hypoglycemia), and can also impair the hormonal response that would normally rectify the low blood sugar. Drinking as little as 2 ounces of alcohol on an empty stomach can lead to very low blood sugar levels. This makes alcohol an even bigger problem for anyone with diabetes. Along with the impact on blood sugar, studies have also shown that alcohol can impact the effectiveness of the hypoglycemic medications, so extreme caution needs to be taken when consuming alcohol by anyone with diabetes.

There is also an increased risk of problems when combining exercise and alcohol. While blood sugars naturally drop during exercise and a body is working on replacing its glycogen stores, consuming alcohol during this time will halt this process and can cause blood sugar levels to stay at an unhealthy level.

The present meaning of alcohol consumption encompasses the entire range of physiological, psychological, social conditions associated therewith, i.e., social drinking, session drinking, binge drinking alcohol abuse, alcohol intoxication and alcoholism.

Symptoms of alcohol consumption include reduced activity in the central nervous system (CNS), loose muscle tone, loss of fine motor coordination, a staggering “drunken” gait, eyes appear “glossy,” pupils may be slow to respond to stimulus, pupils may become constricted, decreased heart rate, lower blood pressure and respiration rate, decreased reflex responses, slower reaction times, skin may be cool to the touch (but the user may feel warm), profuse sweating, loss of fine motor coordination, or odor of alcohol on the breath. Diagnostic criteria for alcohol intoxication include those described in the Diagnostic and Statistical Manual of Mental Disorders, Fourth Edition (DSM-IV).

The term alcohol intoxication as used herein refers to a situation where the quantity of alcohol a person consumes exceeds the individual's tolerance for alcohol and produces, either during or shortly after drinking, clinically important psychological, behavioral or physical abnormalities, such as inappropriate aggression, and impaired judgment and social functioning. One or more of the following signs or symptoms of alcohol intoxication occur shortly after drinking: (1) slurred speech; (2) impaired motor coordination; (3) unsteady gait; (4) nystagmus (involuntary, irregular eye movement characterized by smooth pursuit of an object in one direction and saccadic movement in the other direction); (5) inattention and/or impaired memory; and (6) stupor or coma.

Sobriety, intoxication, alcohol abuse, alcohol-related aggression or alcoholism may be measured according to one or more art recognized tests, such as psychomotor tests, serum alcohol level tests, for example accepted inhalation tests, Diagnostic and Statistical Manual of Mental Disorders, (DSM-IV), Alcohol Abstinence Self-Efficacy Scale, Barratt Impulsiveness Scale −11, State-Trait Anger Expression Inventory −2, Conflict Resolution, Impulsivity and Aggression Questionnaire, Social Problem-Solving Inventory—Revised, Alcohol-Related Aggression Questionnaire, or The Alcohol Use Disorders Identification Test. Levels of alcohol in the body may be measured in urine, blood, breath or saliva.

There is a wide range of variability in blood alcohol levels that different individuals can tolerate without becoming intoxicated. The range may be as great as from 0.3 to 1.5 mg/ml, although most states in the U.S. set the sobriety level for legally driving at 0.8 mg/ml. Some users may develop significant behavioral changes or become intoxicated at a much lower Blood Alcohol Concentration (BAC) than the legal limit. This condition is known as “Alcohol Idiosyncratic Intoxication” or “Pathological Intoxication”. In general, however, the following symptoms are associated with increasing BAC levels:
0.02-0.03 BAC no loss of coordination, slight euphoria and loss of shyness.
0.04-0.06 BAC feeling of well-being, relaxation, lower inhibitions, sensation of warmth, euphoria, some minor impairment of reasoning and memory.
0.07-0.09 BAC slight impairment of balance, speech, vision, reaction time; reduction of judgment and self-control and caution, reason and memory.
0.10-0.125 BAC significant impairment of motor coordination and loss of good judgment; slurred speech; balance, vision, reaction time and hearing are impaired; euphoria. It is illegal to operate a motor vehicle at this level.
0.13-0.15 BAC gross motor impairment and lack of physical control; blurred vision and major loss of balance; euphoria is reduced and dysphoria (anxiety, restlessness) is beginning to appear.
0.16-0.20 BAC dysphoria predominates, nausea may appear.
0.25 BAC the drinker needs assistance in walking; total mental confusion.
0.30 BAC loss of consciousness.
0.40 BAC and up onset of coma, possible death due to respiratory arrest.

The term social drinking refers to the consumption of alcohol in a safe, legal and socially acceptable manner usually without the intent of reaching the point of becoming intoxicated (i.e., to achieve alcohol intoxication). Although the amount of blood alcohol which leads to intoxication varies widely between individuals, three or fewer measured drinks (or a blood alcohol level of up to 0.05%) is generally considered to be within the social drinking range.

The term session drinking refers to drinking in large quantities over a single period of time, or session, without the intention of getting heavily intoxicated. The focus is on the social aspects of the occasion.

The term binge drinking refers to drinking alcohol solely for the purpose of intoxication, although it is quite common for binge drinking to apply to a social situation, creating some overlap in social and binge drinking.

The term alcoholism refers to a primary chronic disease known as alcohol dependence syndrome, the most severe stage of a group of drinking problems. Alcoholism is considered a progressive disease, meaning that the symptoms and effects of drinking alcohol become increasingly more severe over time.

The term alcohol abuse refers to repeated drinking despite alcohol-related physical, social, psychological, or occupational problems (according to DSM IV). When alcohol abuse reaches the alcohol dependence stage, the person may also experience tolerance, withdrawal, and an uncontrolled drive to drink.

As shown in FIG. 9, addition of GC to alcohol protects alcohol-induced liver damage. Thus, the invention further encompasses the use of a composition comprising GC or a combination of GC and CE for protecting from or reducing, ameliorating or attenuating any effect caused by or associated with alcohol consumption. It should be noted that the composition of the invention may be used as add-on to alcoholic beverages as also exemplified herein.

In the context of the present invention, after-effects of alcohol consumption, specifically alcohol hangover, alcohol withdrawal or detoxification are also included, as well as any effect of the alcohol on target organs such as the liver, heart, kidney, brain, muscles, gastrointestinal tract, and any other tissue or organ that can be affected by alcohol or by compounds or states in which the metabolism of alcohol is disturbed.

Alcohol hangover refers to physical and mental symptoms that occur within several hours after alcohol consumption, when a person's BAC is falling, and may continue for up to 24 hours thereafter. Alcohol directly promotes hangover symptoms through its effects on urine production, the gastrointestinal tract, blood sugar concentrations (i.e. hypoglycemia), sleep patterns, and biological rhythms. In addition, effects related to alcohol's absence after a drinking bout (i.e., withdrawal), alcohol metabolism, and other factors (e.g., biologically active, non-alcohol compounds in beverages; the use of other drugs; certain personality traits; and a family history of alcoholism) also may contribute to the hangover condition. The particular set of symptoms experienced and their intensity may vary from person to person and from occasion to occasion. In addition, hangover characteristics may depend on the type of alcoholic beverage consumed and the amount a person drinks.

Physical symptoms of a hangover include fatigue, headache, increased sensitivity to light and sound, redness of the eyes, muscle aches, and thirst. Signs of increased sympathetic nervous system activity can accompany a hangover, including increased systolic blood pressure, rapid heartbeat (i.e., tachycardia), tremor, and sweating. Mental symptoms include dizziness; a sense of the room spinning (i.e., vertigo); and possible cognitive and mood disturbances, especially depression, anxiety, and irritability.

Alcohol-induced hypoglycemia generally occurs after binge drinking over several days in alcoholics who have not been eating. In such a situation, prolonged alcohol consumption, coupled with poor nutritional intake, not only decreases glucose production but also exhausts the reserves of glucose stored in the liver in the form of glycogen, thereby leading to hypoglycemia. Because glucose is the primary energy source of the brain, hypoglycemia can contribute to hangover symptoms such as fatigue, weakness, and mood disturbances. Diabetics are particularly sensitive to the alcohol-induced alterations in blood glucose.

Several lines of evidence suggest that a hangover and mild alcohol withdrawal (AW) share a common biological mechanism. First, the signs and symptoms of hangover and mild AW overlap considerably. Second, it has been known that alcohol re-administration alleviates the unpleasantness of both AW syndrome and hangovers.

In further embodiments, the combined composition of the invention may be applicable for AW and AW syndrome. The AW or AW syndrome or alcohol detoxification, the terms used herein interchangeably, refers to the state following the cessation of excessive drinking, which results from compensatory changes in the CNS that take place in response to chronically administered depressant substances (in this case, alcohol, or more specifically, ethanol). These changes include alterations in the GABA and glutamate receptors, the two main neurotransmitters responsible for inhibitory and excitatory effects. Following chronic alcohol exposure, in an effort to counterbalance alcohol's sedative effects, the body decreases the number or sensitivity of GABA receptors and increases the number or sensitivity of glutamate receptors. When alcohol is removed from the body, however, the CNS and a portion of the sympathetic nervous system that coordinates response to stress remain in an unbalanced “overdrive” state. Sympathetic nervous system hyperactivity accounts for the tremors, sweating, and tachycardia observed in both hangover and AW syndrome.

In more specific embodiments, the composition of the invention is for use in the prevention or alleviation of symptoms or any target organ or tissue damage related to a condition associated with increased or decreased blood alcohol levels following any amount of acute or chronic alcohol drinking, or drinking or injecting or inhaling of any type of substrate that alter the metabolism of alcohol thereby exposing the patient to any type of target organ damage associated with alcohol.

In more specific embodiments, the composition of the invention is for use in the prevention or alleviation of symptoms or any target organ damage related to a condition associated with increased or decreased blood sugar levels. More specifically, such condition may be any one of a hepatic disorder, pancreatic dysfunction, diabetes, obesity, weight gain, states and compounds which alter the insulin resistance state of the body at any level, alcohol intoxication, alcohol withdrawal and vertigo, or any condition associated with alteration of pancreatic or liver function in a way that alter insulin resistance or liver metabolic capability.

In more specific embodiments, the composition of the invention is for use in the prevention or alleviation of symptoms or any target organ damage related drinking, or drinking or injecting or inhaling of any type of substrate that alter the metabolism of alcohol thereby exposing the patient to any type of target organ damage associated with alcohol or sugar, or altering the state of the insulin resistance of the patient.

In yet some other embodiments, the composition of the invention may be applicable for use in a method for treating a subject suffering from a disorder associated with increased or decreased blood sugar levels.

In more specific embodiments, such disorder may be any one of a hepatic disorder, pancreatic dysfunction, diabetes, obesity, insulin resistance, metabolic syndrome, alcohol intoxication, alcohol withdrawal and vertigo, any type of inflammation of the pancreas, liver, muscle or the adipose tissue.

In further embodiments, the composition of the invention may optionally further comprise additional therapeutic agent, wherein said additional therapeutic agent is any one of insulin, N-acetyl cysteine (NAC), any immuno-modulatory antibody, thiamine (vitamin B1), a benzodiazepine or any combination thereof.

Insulin is a peptide hormone, produced by beta cells in the pancreas, and is central to regulating carbohydrate and fat metabolism in the body. It causes cells in the skeletal muscles, and fat tissue to absorb glucose from the blood. In other words, insulin is an anabolic hormone causing cells to take up energy substrates at the times of excess. Insulin acts through a complex mechanism involving protein phosphorylation and dephosphorylation, which lead to controlled activation of glycogen synthetase and pyruvate dehydrogenase and inactivation phosphofructokinase II and hormone-sensitive lipase. Complicated control mechanism steer hormone secretion such that metabolism is constantly adjusted by hormones to meet our widely varying energy intake and expenditure, assuring a constant internal milieu. Insulin action is countered by the catabolic hormones glucagon, adrenalin, noradrenalin and growth hormone, which act primarily through cyclic AMP (cAMP) and protein kinase A.

Supplementation of exogenous insulin (most commonly injected subcutaneously) is the predominant therapy for patients with type 1 diabetes (which do not produce insulin). Medical preparations of insulin (from the major suppliers—Eli Lilly, Novo Nordisk, and Sanofi Aventis—or from any other) are specially prepared mixtures of insulin plus other substances including preservatives. These delay absorption of the insulin, adjust the pH of the solution to reduce reactions at the injection site. Most of the medical insulin produces today is recombinant insulin, which almost completely replaced insulin obtained from animal sources (e.g. pigs and cattle). A variety of different recombinant human insulin preparations are in widespread use. Since 2003, yeast-based insulin also became available for medical use. In addition, a number of insulin analogues have been developed, which retain the hormone's glucose management functionality. They are either absorbed rapidly in an attempt to mimic real beta cell insulin (as with Lilly's lispro, Novo Nordisk's aspart and Sanofi Aventis' glulisine), or steadily absorbed after injection instead of having a ‘peak’ followed by a more or less rapid decline in insulin action (as with Novo Nordisk's version Insulin detemir and Sanofi Aventis's Insulin glargine), all while retaining insulin's glucose-lowering action in the human body.

The major problem with management of insulin therapy is choosing the most appropriate insulin type and dosage/timing for each diabetic patient.

The commonly used types of insulin are:
fast-acting using insulin analogues aspart, lispro, and glulisine, which begin to work within 5 to 15 minutes and are active for 3 to 4 hours.
short-acting using regular insulin which begins working within 30 minutes and is active about 5 to 8 hours.
intermediate-acting using NPH insulin which begins working in 1 to 3 hours and is active 16 to 24 hours.
long acting using analogues glargine and detemir, each of which begins working within 1 to 2 hours and continue to be active, without major peaks or dips, for about 24 hours.
ultra-long acting currently only including the analogue degludec, which begins working within 30-90 minutes, and continues to be active for greater than 24 hours.
combination insulin products using either fast-acting or short-acting insulin with a longer acting insulin, typically an NPH insulin.

It must be understood that the invention encompasses the use of any insulin preparation as an additional therapeutic agent in any of the pharmaceutical compositions described herein.

Oral, intradermal, intrarectal, inhaled, intrapulmonary, or intramucosl administration of insulin or of compounds that alter the insulin metabolism or that alter or potentiate its effects, whether via a direct effect following systemic absorption or an indirect effect following an effect on the gut associated lymphoid tissue, or any subset of cells with which they are in direct contact, may exert a beneficial effect on the glucose metabolism. It also has a beneficial effect on targets of the metabolic syndrome such as fatty liver disease, NASH, atherosclerosis, heart disease, hyperlipidemia and diabetes.

Still further, in certain embodiments, the additional therapeutic agent may be NAC. N-acetyl cysteine (Brand names: NAC, Mucomyst, Acetadote) has many uses as medicine. NAC is used to counteract acetaminophen (Tylenol) and carbon monoxide poisoning. It is also used for chest pain (unstable angina), bile duct blockage in infants, amyotrophic lateral sclerosis (ALS, Lou Gehrig's disease), Alzheimer's disease, allergic reactions to the anti-seizure drug phenytoin (Dilantin), and an eye infection called keratoconjunctivitis. It is also used for reducing levels of a type of cholesterol called lipoprotein (a), homocysteine levels (a possible risk factor for heart disease) and the risk of heart attack and stroke in patients with serious kidney disease. Some people use NAC for chronic bronchitis, chronic obstructive pulmonary disease (COPD), hay fever, a lung condition called fibrosing alveolitis, head and neck cancer, and lung cancer. It is also used for treating some forms of epilepsy; ear infections; complications of kidney dialysis; chronic fatigue syndrome (CFS); an autoimmune disorder called Sjogren's syndrome; preventing sports injury complications; radiation treatment; increasing immunity to flu and H1N1 (swine) flu; and for detoxifying heavy metals such as mercury, lead, and cadmium.

Specifically relevant to the present context, NAC is also used for preventing alcoholic liver damage; for protecting against environmental pollutants including carbon monoxide, chloroform, urethanes and certain herbicides; for reducing toxicity of ifosfamide and doxorubicin, drugs that are used for cancer treatment; as a hangover remedy; for preventing kidney damage due to certain X-ray dyes; and for human immunodeficiency virus (HIV).

Healthcare providers give NAC intravenously (IV) for acetaminophen overdose, acrylonitrile poisoning, amyotrophic lateral sclerosis (ALS, Lou Gehrig's disease), kidney failure in the presence of liver disease (hepatorenal syndrome), chest pain in combination with nitroglycerin, heart attack in combination with nitroglycerin and streptokinase, and for helping to prevent multi-organ failure leading to death. NAC is sometimes inhaled or delivered through a tube in the throat to treat certain lung disorders such as pneumonia, bronchitis, emphysema, cystic fibrosis, and others.

Still further, in certain embodiments, the additional therapeutic agent may bean immuno-modulatory antibody being administered orally, intravenously, intrarectaly, by inhalation or intradermally. Such antibodies may include, but are not limited to, anti TNF antibodies, both, chimeric or humanized, anti integrin antibodies, or any type of antibody. These antibodies may be combined with the combined composition of the invention and/or with any of the above compounds for prevention or amelioration of toxicity or unwanted side effects of sugar, alcohol or any drug. Alternatively, these antibodies may be combined with the compositions of the invention and/or any of the compounds described above for augmenting the beneficial effects of these antibodies or of any of the compounds described herein above.

In yet another embodiment, an additional therapeutic agent may be Vitamin B1. Vitamin B1 (also thiamine or thiamin, i.e. sulfur-containing vitamin) is a water-soluble vitamin of the B complex. Its phosphate derivatives are involved in many cellular processes. The best-characterized form is thiamine pyrophosphate (TPP), a coenzyme in the catabolism of sugars and amino acids. Thiamine is used in the biosynthesis of the neurotransmitter acetylcholine and gamma-aminobutyric acid (GABA). Vitamin B is synthesized only in bacteria, fungi, and plants, animals must obtain it from their diet, and thus, for them, it is an essential nutrient. In mammals, deficiency results in Korsakoff's syndrome, optic neuropathy and Beriberi disease that affects the peripheral nervous system (polyneuritis) and/or the cardiovascular system. Thiamine deficiency has a potentially fatal outcome if it remains untreated. In less severe cases, nonspecific signs include malaise, weight loss, irritability and confusion.

Specifically in this context, alcoholics may have thiamine deficiency because of:
inadequate nutritional intake.
active transport of thiamine into enterocytes is disturbed during acute alcohol exposure.
liver thiamine stores are reduced due to hepatic steatosis or fibrosis.
impaired thiamine utilization due to chronic alcohol consumption.
ethanol per se inhibits thiamine transport in the gastrointestinal system.

Vitamin B1 supplementation is one of the therapeutic approaches to AW syndrome. Following improved nutrition and the removal of alcohol consumption, some impairments linked with thiamine deficiency are reversed, in particular poor brain functionality.

Benzodiazepines, that may also serve as an additional therapeutic agent, (sometimes colloquially benzo, often abbreviated BZD) is a psychoactive drug whose core chemical structure is the fusion of a benzene ring and a diazepine ring, the most notable example of which is Valium. BZD enhance the effect of the neurotransmitter GABA at the GABAA receptor, resulting in sedative, hypnotic (sleep-inducing), anxiolytic (anti-anxiety), euphoric, anticonvulsant, and muscle relaxant properties; also seen in the applied pharmacology of high doses of many shorter-acting BZD are amnesic-dissociative actions. These properties make BZD useful in treating anxiety, insomnia, agitation, seizures, muscle spasms, AW and as a premedication for medical or dental procedures.

In some alternative embodiments, the combined composition of the invention may be particularly useful in methods for treating, preventing, ameliorating, reducing or delaying the onset of an immune-related disorder.

In more specific embodiments, such immune-related disorder may be any one of an inflammatory disorder, an autoimmune disorder, an infectious disease and a proliferative disorder.

In some specific embodiments, the combined composition of the invention may be suitable for treating an immune-related disorder, for example, hepatitis.

In some embodiments, the composition of the invention may further comprise at least one additional therapeutic agent. More specifically, such agent may be any one of insulin, antibodies directed to inflammatory cytokine, or antibodies such as anti TNF antibodies, statins, analgesics, chemotherapeutic agents and antibiotics.

Still further embodiments relate to the combined composition of the invention for use in a method for treating liver damage in a subject in need thereof. More specifically, such composition comprises a therapeutically effective amount of a natural or synthetic beta-glycolipid and polyethoxylated castor oil or any derivative thereof, or any combination thereof.

In more specific embodiments, such subject may be a subject suffering from a liver disease, that may be any one of viral, alcoholic or autoimmune hepatitis, alcoholic or autoimmune cirrhosis, alcoholic fatty liver disease, non alcoholic fatty liver disease (NAFLD), any type of liver steatosis, for example, due to other disease such as Wilson's disease or alpha 1 anti trypsin deficiency, nonalcoholic steatohepatits (NASH), hepatocellular carcinoma, drug-induced liver disease and pediatric liver disease and any type of metabolic liver disease, for example, glycogen storage disease

In certain embodiments, the composition of the invention may optionally further comprise at least one additional therapeutic agent. More specifically, such additional therapeutic agent may be any one of insulin, NAC, vitamin B1, a benzodiazepine, an anti-viral or anti-inflammatory drug, a chemotherapeutic agent.

In further alternative embodiments, the composition of the invention may be applicable for use in a method for treating, preventing, ameliorating, reducing or delaying the onset of acute or chronic toxic effect of a drug or any type of side effect of any drug, whether due to a direct or indirect effect of the drug or any of its metabolites.

The protective effect of the combined composition of the invention enables the preparation of “safe drugs” comprising the protective composition of the invention. A combined composition further comprising in addition to the GC and Cremophore EL, or alternatively, a combination of GC and polyethylene glycol or with beta cyclo dextrin, or any combination thereof, to any type of therapeutic compound or food, or any ingredient, provides protection against unwanted side effects, provides protection against any type of target organ toxicity, and prevent any type of toxicity or side effect of said drugs. In addition, the combined composition of the invention may enhance and augment the beneficial effect of the drug or compound either by exerting an additive effect or a synergistic effect. This beneficial effect may act via augmenting of the mechanism of action of the drug, or compound, or via an indirect adjuvant effect, for example by activating other pathways, cells or organs.

According to one specific embodiment, the invention provides a combined composition further comprising in addition to the GC and Cremophore EL, also at least one additional therapeutic agent selected from analgesic or antipyretic drug. Such analgesic or antipyretic drug may be according to certain embodiments, an inducer or inhibitor of Cytochrom P-450 selected from the group consisting of: Acetaminophen, Phenobarbital, Phenytoin, Carbamazepine, Primidone, Ethanol, Glucocorticoids, Rifampin, Griseofulvin, Quinine, Omeprazole, Amiodarone, Cimetidine, Erythromycin, Grape fruit, Isoniazid, Ketoconazole, Metronidazole, Sulfonamides, Chlorpromazine, phenylbutazone, halogenated anesthetic agents, sulindac, Dapsone, INH, halothane, amoxicillin-clavulanic acid, phenobarbital, Para-amino salicylate, Clofibrate, Procainamide, Gold salts, propylthiouracil, chloramphenicol, nitrofurantoin, methoxyflurane, penicillamine, paraquat, Tetracycline, Contraceptive and anabolic steroids, rifampin, Aspirin and Sodium valproate. According to one specific embodiment, the invention relates to a combined composition comprising GC, Cremophore EL and acetaminophen, thereby providing a safe preparation of acetaminophen, having reduced potential for hepatic toxicity.

More specifically, the invention further provides a pharmaceutical composition for treating, preventing, ameliorating, reducing or delaying the onset of acute or chronic toxic effect of an analgesic or an antipyretic drug in a subject in need thereof. Moreover, the pharmaceutical composition of the invention may be used for treating and preventing any type of liver insult selected from infectious metabolic, toxic, immune, or perfusion or blood flow related hepatic injury. The pharmaceutical composition of the invention comprises as an active ingredient a therapeutically effective amount of a combination of at least one natural or synthetic beta-glycolipid and at least one polyethoxylated castor oil or any derivatives thereof, specifically, Cremophore EL, and optionally at least one additional therapeutic agent, with a pharmaceutically acceptable carrier. In more specific embodiments the drug may be an analgesic or an antipyretic drug.

Such drug may be according to certain embodiments, an inducer or inhibitor of Cytochrom P-450 selected from the group consisting of: Acetaminophen, Phenobarbital, Phenytoin, Carbamazepine, Primidone, Ethanol, Glucocorticoids, Rifampin, Griseofulvin, Quinine, Omeprazole, Amiodarone, Cimetidine, Erythromycin, Grape fruit, Isoniazid, Ketoconazole, Metronidazole, Sulfonamides, Chlorpromazine, phenylbutazone, halogenated anesthetic agents, sulindac, Dapsone, INH, halothane, amoxicillin-clavulanic acid, phenobarbital, Para-amino salicylate, Clofibrate, Procainamide, Gold salts, propylthiouracil, chloramphenicol, nitrofurantoin, methoxyflurane, penicillamine, paraquat, Tetracycline, Contraceptive and anabolic steroids, rifampin, Aspirin and Sodium valproate.

According to one specific embodiment, the pharmaceutical composition of the invention is intended for treating, preventing, ameliorating, reducing or delaying the onset of acute or chronic toxic effect of the analgesic drug N-(4-hydroxyphenyl)ethanamide, known as acetaminophen (paracetamol).

N-(4-hydroxyphenyl) ethanamide Paracetamol or acetaminophen is a widely used over-the-counter analgesic (pain reliever) and antipyretic (fever reducer). It is commonly used non-steroidal analgesic agent for the relief of fever, headaches, and other minor aches and pains, and is a major ingredient in numerous cold and flu remedies.

While acetaminophen has fewer gastro-intestinal side effects than aspirin, another commonly used non-steroidal analgesic agent, acute and chronic acetaminophen toxicity can result in gastro-intestinal symptoms, severe liver damage, and even death. The precise intermediates in the acetaminophen toxic metabolite pathway are not yet known. As indicated herein before, it had been thought that when acetaminophen was ingested, the cytochrome P-450 dependent enzyme system of the liver produced a potentially toxic metabolite of acetaminophen which was the cause of acetaminophen toxicity.

It was further believed that when safe amounts of acetaminophen had been ingested, this toxic metabolite was cleared by hepatic glutathione stores. However in the case of acute or chronic overdose, excessive levels of the toxic metabolite were thought to delete the glutathione stores in the liver, resulting in hepatic necrosis. Later studies have proposed that acetaminophen induced hepatic necrosis may be due to cellular oxidative stress, resulting both in lipid peroxidation, protein and non-protein thiol oxidation, and changes in the intracellular calcium homeostasis. Symptoms of acute acetaminophen toxicity are typically mild or non-existent until at least 48 hours post-ingestion.

Thus, in yet another embodiment the acute or chronic toxic effect of acetaminophen treated by the combined composition of the invention may be any one of drug induced liver injury (DILI), drug-induced acute steatosis, cytotoxic hepatocellular injury, acute liver failure (ALF), reperfusion injury, ischemic liver disease and acute cholestatic injury.

According to one specific embodiment, the pharmaceutical combined composition of the invention is particularly applicable for treating, preventing, ameliorating, reducing or delaying the onset of drug induced liver injury (DILI), caused by acetaminophen.

It should be appreciated that the different Cytochrome P-450 inducing or inhibiting drugs may lead to different hepatic injuries, and therefore, may be prevented or treated by the combined compositions of the invention. For example, chlorpromazine, phenylbutazone, halogenated anesthetic agents and sulindac may cause fever, rash and eosinophilia. Dapsone may lead to sulfone syndrome (i.e., fever, rash, anemia, and jaundice), INH (Isoniazid (Laniazid, Nydrazid), also known as isonicotinylhydrazine (INH) and halothane may cause acute viral hepatitis, Chlorpromazine, erythromycin, amoxicillin- and clavulanic acid may lead to obstructive jaundice. Phenytoin, carbamazepine, Phenobarbital and primidone may cause anticonvulsant hypersensitivity syndrome (i.e., triad of fever, rash, and liver injury), Para-amino salicylate, phenytoin, sulfonamides, may lead to serum sickness syndrome, Clofibrate may lead to Muscular syndrome (i.e., myalgia, stiffness, weakness, elevated creatine kinase level), Procainamide may cause Antinuclear antibodies (ANAs), Gold salts, propylthiouracil, chlorpromazine and chloramphenicol may cause marrow injury. Drugs such as Amiodarone and nitrofurantoin may be lead to associated pulmonary injury and Gold salts, methoxyflurane, penicillamine, paraquat may also lead to Associated renal injury. Tetracycline may cause Fatty liver of pregnancy, Contraceptive and anabolic steroids and rifampin may cause bland jaundice, Aspirin may cause Reye syndrome, and Sodium valproate may lead to Reye like syndrome.

Still further, other acute hepatocellular injuries caused by drugs may be treated or prevented by the combined composition of the invention. For example, acute viral hepatitis-like picture may be caused by INH, halothane, diclofenac and troglitazone. Mononucleosis like picture may be a result of using phenytoin, sulfonamides or dapsone. Chronic hepatocellular injury may be a result of Pemoline or methyldopa. Massive necrosis may be a result of using acetaminophen, halothane or diclofenac.

Steatosis may also be a result of using different drugs, for example, Macro vesicular steatosis may be caused by Alcohol, methotrexate, corticosteroids, minocycline, nifedipine and TPN, Microvesicular steatosis may be caused by alcohol, valproic acid, tetracycline and piroxicam. Steatohepatitis may be a result of Amiodarone, nifedipine, synthetic estrogens and didanosine. Pseudoalcoholic injury may be caused by Amiodarone, Acute cholestasis maybe a result of using Amoxicillin-clavulanic acid, erythromycin and sulindac. Chronic cholestasis may be caused by Chlorpromazine, sulfamethoxazole-trimethoprim, tetracycline or ibuprofen. Granulomatous hepatitis may be a result of using Carbamazepine, allopurinol and hydralazine. Vascular injury may be caused by steroids, Neoplasia may be a result of using Contraceptives or anabolic steroids. Adenoma may be caused by steroids, Angiosarcoma may be a result of Vinyl chloride. Hepatocellular carcinoma may be caused by Anabolic steroids, aflatoxin, arsenic or vinyl chloride.

More particularly, a drug such as Amoxicillin may cause Hepatic dysfunction including jaundice, hepatic cholestasis, and acute cytolytic hepatitis.

Statins are among the most widely prescribed medications in the western world. The use of statins/HMG-CoA reductase inhibitors is associated with biochemical abnormalities of liver function, and thus may be also prevented or treated by the combined composition of the invention. Moderate elevations of serum transaminase levels (<3 times the upper limit of the reference range) have been reported following initiation of therapy and are often transient. Elevations are not accompanied by any symptoms and do not require interruption of treatment. Persistent increases in serum transaminase levels (>3 times the upper limit of the reference range) occur in approximately 1% of patients, and these patients should be monitored until liver function returns to normal after drug withdrawal. Active liver disease or unexplained transaminase elevations are contraindications to use of these drugs. Patients with a recent history of liver disease or persons who regularly consume alcohol in large quantities, should use statins in a regulated manner.

In certain embodiments, the combined compositions of the invention may also be applicable for preventing and treating liver injury caused by Rifampin. Rifampin is usually administered with INH. On its own, rifampin may cause mild hepatitis, but this is usually in the context of a general hypersensitivity reaction. Fatalities associated with jaundice have occurred in patients with liver disease and in patients taking rifampin with other hepatotoxic agents. Careful monitoring of liver function (especially SGPT/SGOT) should be performed prior to therapy and then every 2-4 weeks during therapy. In some cases, hyper-bilirubinemia resulting from competition between rifampin and bilirubin for excretory pathways of the liver can occur in the early days of treatment. Isolated cholestasis also may occur.

In yet a further embodiment, the combined composition of the invention may be applicable for preventing or treating liver damage caused by Valproic acid and divalproex sodium. More specifically, microvesicular steatosis is observed with alcohol, aspirin, valproic acid, amiodarone, piroxicam, stavudine, didanosine, nevirapine, and high doses of tetracycline. Prolonged therapy with methotrexate, INH, ticrynafen, perhexiline, enalapril, and valproic acid may lead to cirrhosis. Valproic acid typically causes microsteatosis. This drug should not be administered to patients with hepatic disease and may be used with caution in patients with a prior history of hepatic disease. Those at particular risk include children younger than 2 years, those with congenital metabolic disorders or organic brain disease, and those with seizure disorders treated with multiple anticonvulsants.

Hepatic failures resulting in fatalities have occurred in patients receiving valproic acid. These incidents usually occur during the first six months of treatment and are preceded by nonspecific symptoms such as malaise, weakness, lethargy, facial edema, anorexia, vomiting, and even loss of seizure control.

It should be further appreciated that the combined composition of the invention may also be used for preventing or treating liver damage caused by using herbs. The increasing use of alternative medicines has led to many reports of toxicity. The spectrum of liver disease is wide with these medicines, for example: Senecio/crotalaria (Bush teas) can cause venoocclusive disease. Germander in teas is used for its anticholinergic and antiseptic properties. Jaundice with high transaminase levels may occur after two months of use, but it disappears after stopping the drug. Chaparral is used for a variety of conditions, including weight loss, cancer, and skin conditions. It may cause jaundice and fulminant hepatic failure. Chinese herbs have also been associated with hepatotoxicity.

According to certain embodiments, the combined composition of the invention may also be applicable in treating liver damage caused by recreational drugs. More specifically, Ecstasy is an amphetamine used as a stimulant and may cause hepatitis and cirrhosis. Cocaine abuse has been associated with acute elevation of hepatic enzymes. Liver histology shows necrosis and microvascular changes.

More specifically, according to some embodiments, in addition to the enhancement or the augmentation of the beneficial effect of insulin whether via a direct or an indirect adjuvant effect, as described above, the pharmaceutical composition of the invention is intended for treating, preventing, ameliorating, reducing or delaying the onset of acute or chronic toxic effect of insulin.

According to certain specific embodiments, the composition of the invention is particularly suitable for oral or mucosal administration. The usefulness of an oral formulation requires that the active agent or combinations of the invention be bioavailable.

Bioavailability of orally administered drugs can be affected by a number of factors, such as drug absorption throughout the gastrointestinal tract, stability of the drug in the gastrointestinal tract, and the first pass effect. Thus, effective oral delivery of an active agent or combination requires that the active agent have sufficient stability in the stomach and intestinal lumen to pass through the intestinal wall. Many drugs, however, tend to degrade quickly in the intestinal tract or have poor absorption in the intestinal tract so that oral administration is not an effective method for administering the drug.

More specifically, the composition of the invention may be suitable for mucosal administration, for example, pulmonary, buccal, nasal, intranasal, sublingual, rectal, dermal, vaginal administration and any combination thereof.

Although preferred administration is oral or mucosal, it should be appreciated that the composition of the invention may be also suitable for intravenous, intramuscular, subcutaneous, intraperitoneal, perenteral, transdermal, sublingual, topical, administration, or any combination thereof.

In another aspect, the invention further relates to an oral or mucosal pharmaceutical composition made by combining a therapeutically effective amount of at least one natural or synthetic beta-glycolipid and at least one polyethoxylated castor oil or any derivative thereof, and optionally at least one additional therapeutic agent, with a pharmaceutically acceptable carrier.

In some embodiments, the oral or mucosal composition of the invention may comprise as an active ingredient GC and at least one adjuvant, for example, PEG and/or beta cyclo dextrin, or any combination thereof.

According to a specifically preferred embodiment, such composition is as described by the invention. Pharmaceutical compositions suitable for oral administration are typically solid dosage forms (e.g., tablets) or liquid preparations (e.g., solutions, suspensions, or elixirs).

Solid dosage forms are desirable for ease of determining and administering dosage of active ingredient, and ease of administration, particularly administration by the subject at home.

Liquid dosage forms also allow subjects to easily take the required dose of active ingredient. Liquid preparations can be prepared as a drink, or to be administered, for example, by a naso gastric tube (NG tube). Liquid oral pharmaceutical compositions generally require a suitable solvent or carrier system in which to dissolve or disperse the active agent, thus enabling the composition to be administered to a subject. A suitable solvent system is compatible with the active agent and non-toxic to the subject. Typically, liquid oral formulations use a water-based solvent.

The oral compositions of the invention can also optionally be formulated to reduce or avoid the degradation, decomposition, or deactivation of the active agents by the gastrointestinal system, e.g., by gastric fluid in the stomach. For example, the compositions can optionally be formulated to pass through the stomach unaltered and to dissolve in the intestines, i.e., enteric coated compositions.

As indicated above, the combined beta-glycolipids and polyethoxylated castor oil or any derivative thereof described herein, or any combination of GC with an adjuvant such as PEG and/or beta cyclo dextrin can be incorporated as active ingredients into a pharmaceutical composition suitable for oral or mucosal administration, e.g., by ingestion, inhalation, or absorption, e. g., via nasal, intranasal, pulmonary, buccal, sublingual, rectal, dermal, or vaginal administration. Such compositions can include an inert diluent or an edible carrier. For the purpose of oral therapeutic administration, the active compound Cremophore EL and a beta-glucosylceramide (GC) can be incorporated with recipients and used in solid or liquid (including gel) form. Oral compositions can also be prepared using an excipient. Pharmaceutically compatible binding agents, and/or adjuvant materials can be included as part of the composition. Oral dosage forms comprising combined beta-glycolipid and Cremophore EL are provided, wherein the dosage forms, upon oral administration, provide a therapeutically effective blood level of the combined beta-glycolipid and Cremophore EL to a subject. Also provided are mucosal dosage forms comprising said combination wherein the dosage forms, upon mucosal administration, provide a therapeutically effective blood level of the combined beta-glycolipid and Cremophore EL to a subject. For the purpose of mucosal therapeutic administration, the active combined compounds (e.g., beta-glucosylceramide with Cremophore EL, or GC with PEG or with beta cyclo dextrin) can be incorporated with excipients or carriers suitable for administration by inhalation or absorption, e.g., via nasal sprays or drops, or rectal or vaginal suppositories.

Solid oral dosage forms include, but are not limited to, tablets (e.g., chewable tablets), capsules, caplets, powders, pellets, granules, powder in a sachet, enteric coated tablets, enteric coated beads, and enteric coated soft gel capsules. Also included are multi-layered tablets, wherein different layers can contain different drugs. Solid dosage forms also include powders, pellets and granules that are encapsulated. The powders, pellets, and granules can be coated, e.g., with a suitable polymer or a conventional coating material to achieve, for example, greater stability in the gastrointestinal tract, or to achieve a desired rate of release.

In addition, a capsule comprising the powder, pellets or granules can be further coated. A tablet or caplet can be scored to facilitate division for ease in adjusting dosage as needed.

The dosage forms of the present invention can be unit dosage forms wherein the dosage form is intended to deliver one therapeutic dose per administration, e.g., one tablet is equal to one dose. Such dosage forms can be prepared by methods of pharmacy well known to those skilled in the art. Typical oral dosage forms can be prepared by combining the active ingredients in an intimate admixture with at least one excipient according to conventional pharmaceutical compounding techniques. Excipients can take a wide variety of forms depending on the form of preparation desired for administration. For example, excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents. Examples of excipients suitable for use in oral liquid dosage forms include, but are not limited to, water, glycols, oils, alcohols, flavoring agents, preservatives, and coloring agents. Tablets and capsules represent convenient pharmaceutical compositions and oral dosage forms, in which case solid excipients are employed. If desired, tablets can be coated by standard aqueous or non-aqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

As one example, a tablet can be prepared by compression or by molding. Compressed tablets can be prepared, e.g., by compressing, in a suitable machine, the active ingredients (e.g., combined beta-glycolipid and Cremophore EL, or GC with PEG or with beta cyclo dextrin) in a free-flowing form such as powder or granules, optionally mixed with an excipient. Molded tablets can be made, e.g., by molding, in a suitable machine, a mixture of the powdered combined beta-glycolipid and Cremophore EL compound moistened, e.g., with no inert liquid diluent.

A second aspect of the invention relates to a method for controlling blood sugar levels in a subject, treating an immune related disorder, treating liver damage and for treating, preventing, ameliorating, reducing or delaying the onset of acute or chronic toxic effect of a drug. In more specific embodiments, the method of the invention comprises providing to a subject a food supplement comprising as an active ingredient at least one of: (a) at least one natural or synthetic beta-glycolipid or any derivative/s thereof; (b) at least one polyethoxylated castor oil or any derivative thereof; (c) at least one adjuvant selected from PEG or beta cyclo dextrin or any derivative/s thereof (d) any combination of (a), (b) and (c); and (e) a composition comprising any one of (a), (b), (c) and (d).

In some embodiments, the natural or synthetic beta-glycolipid comprise used by the method of the invention may be any one of a glycosphingolipid, glucosylceramide, monosaccharide ceramide, galatosylceremide, lactosylceramide, gal-gal-glucosyl-ceramide, GM2 ganglioside, GM3 ganglioside, globoside or any soy derivative or a combination thereof.

According to more specific embodiments, the glucosylceramide used by the method of the invention may be a beta glucosylceramide (GC).

In yet some further embodiments, the derivative of polyethoxylated castor oil used by the method of the invention may be Cremophore EL.

In some alternative embodiments, the method of the invention may use a combination of GC or any derivatives thereof with PEG or any derivatives thereof.

In yet some other embodiments, the methods of the invention may use a combination of GC and cyclo dextrin or any derivative/s thereof.

In certain embodiments the composition used by the method of the invention may be formulated in a formulation adapted for add-on to a solid, semi-solid or liquid food, beverage, food additive, food supplement, medical food, drug and/or any type of pharmaceutical compound.

In some embodiments, the composition used by the method of the invention may be adapted for add-on a food and/or beverage that comprise an increased content of sugar and/or alcohol or to a food or beverage that may be associated with increase in blood sugar or alcohol level via alteration of the insulin resistance state or the capability to alter alcohol metabolism by the body. In some embodiments, the method of the invention is applicable for controlling blood sugar levels in a subject. More specifically, such control may be inhibiting increase or decrease in blood sugar levels or alternatively, altering the insulin resistance state in the treated subject.

In more specific embodiments, the method of the invention may be applicable in the prevention or alleviation of symptoms related to a condition associated with increased or decreased blood sugar levels. More specifically, such condition may be any one of a hepatic disorder, pancreatic dysfunction, diabetes, obesity, weight gain, alcohol intoxication, alcohol withdrawal and vertigo, or any condition associated with alteration of pancreatic or liver function in a way that alter insulin resistance or liver metabolic capability.

In yet some other embodiments, the method of the invention is adapted for treating a subject suffering from a disorder associated with increased or decreased blood sugar levels.

In more specific embodiments, such disorder may be any one of a hepatic disorder, pancreatic dysfunction, diabetes, obesity, insulin resistance, metabolic syndrome, alcohol intoxication, alcohol withdrawal and vertigo, any type of inflammation of the pancreas, liver, muscle or the adipose tissue, any type of target organ or tissue damage that is directly or indirectly related to consumption of alcohol, sugar, or any of their derivatives by acting by themselves of via activation of other molecules or pathways.

The present invention is directed at treating, controlling or preventing a number of medical conditions. In general, the terms preventing, controlling and treating encompass a range of conditions, starting from prevention of the development of a disease or a symptom in a patient who may predisposed to a disease but has yet been diagnosed; further including reduction, retardation or inhibition of progression symptoms of a disease; and also alleviation of symptoms of an already existing disease, i.e. reversal of said symptoms.

Methods and compositions of the invention are specifically relevant to treating, controlling, ameliorating, or preventing body weight gain, obesity, metabolic syndrome and diabetes.

By body weight gain is meant specifically body fat gain that is maintained or decreased by applying the methods and compositions of the invention. A decrease in weight or body fat may protect against cardiovascular disease by lowering blood pressure, total cholesterol, LDL cholesterol and triglycerides, and may alleviate symptoms associated with chronic conditions such as hypertension, coronary heart disease, type 2 diabetes, osteoarthritis, sleep apnea and degenerative joint disease.

The present invention is applicable to all types of obesity, including endogenous obesity, exogenous obesity, hyper-insulinar obesity, hyperplastic-hypertrophic obesity, hypertrophic obesity, hypothyroid obesity and morbid obesity. Moreover, inflammation-mediated obesity may be treated particularly effectively in accordance with the invention.

By metabolic syndrome, or syndrome X, is meant a complex multi-factorial condition accompanied by an assortment of abnormalities including hypertension, hyper-triglyceridemia, hyperglycemia, low high-density lipoprotein (HDL) cholesterol and abdominal obesity, which, among others, may lead to pro-thrombotic (e.g., elevated fibrinogen or plasminogen activator inhibitor-1 in the blood) and pro-inflammatory (e.g., elevated C-reactive protein (CRP) in the blood) conditions.

The World Health Organization (WHO) guidelines for diagnosis of metabolic syndrome are (Journal of Hypertension, Volume 17, pages 151-183, 1999):
hypertension (>140 mm Hg systolic or >90 mm Hg diastolic).
dyslipidemia, defined as elevated plasma triglycerides (150 mg/dL), and/or low high-density lipoprotein (HDL) cholesterol (<35 mg/dL in men, <39 mg/dL in women).
visceral obesity defined as a high body mass index (BMI) (30 kg/m2) and/or a high waist-to-hip ratio (>0.90 in men, >0.85 in women).
microalbuminuria (urinary albumin excretion rate of 20 g/min).

Alternatively, according to the National Cholesterol Education Program (NCEP) metabolic syndrome if at least three of the following five symptoms are present (JAMA, Volume 285, pages 2486-2497, 2001):
waist circumference >102 cm (40 in) for men or >88 cm (37 in) for women.
triglyceride level of 150 mg/dL.
HDL cholesterol level <40 mg/dL for men or <50 mg/dL for women.
blood pressure >130/85 mm Hg.
fasting glucose >110 mg/dL.

Each of the disorders associated with metabolic syndrome are risk factors in their own right, and can promote atherosclerosis, cardiovascular disease, stroke, and other adverse health consequences. However, when present together, these factors are predictive of increased risk of cardiovascular disease and stroke.

In the context of the present invention, controlling or treating metabolic syndrome using the combined compositions of the invention, is meant reducing severity and/or number of symptoms associated with this medical condition, i.e. reducing any one of elevated blood glucose, glucose intolerance, insulin resistance, elevated triglycerides, elevated LDL-cholesterol, low HDL cholesterol, elevated blood pressure, abdominal obesity, pro-inflammatory states, and pro-thrombotic states. Additionally or alternatively, it is meant reducing the risk and/or the onset of developing associated diseases, i.e. cardiovascular disease, coronary heart disease and other diseases related to plaguing of the artery walls and diabetic conditions.

Further, methods and compositions of the invention are particularly advantageous for treating, controlling and preventing diabetes or diabetic conditions, such as type 1 diabetes, type 2 diabetes, gestational diabetes, pre-diabetes, slow onset autoimmune diabetes type 1 (LADA), hyperglycemia or any type of condition or compound that expose the patient to pre diabetes or to diabetes or that alters the stage of insulin resistance. For the purposes of treatment, the diabetes may be overt, diagnosed diabetes, e.g., type 2 diabetes, or a pre-diabetic condition.

Diabetes mellitus (generally referred to herein as diabetes) is a disease that is characterized by impaired glucose regulation. Diabetes is a chronic disease that occurs when the pancreas fails to produce enough insulin or when the body cannot effectively use the insulin that is produced, resulting in an increased concentration of glucose in the blood (hyperglycemia). The WHO recognizes three main forms of diabetes mellitus: type 1, type 2, and gestational diabetes (occurring during pregnancy), which have different causes and population distributions. While, ultimately, all forms are due to the beta cells of the pancreas being unable to produce sufficient insulin to prevent hyperglycemia, the causes are different. Type 1 diabetes is usually due to autoimmune destruction of the pancreatic beta cells. Type 2 diabetes is characterized by insulin resistance in target tissues, this causes a need for abnormally high amounts of insulin and diabetes develops when the beta cells cannot meet this demand. Gestational diabetes is similar to type 2 diabetes in that it involves insulin resistance, hormones in pregnancy may cause insulin resistance in women genetically predisposed to developing this condition.

Type 1 diabetes is also recognized as insulin-dependent, juvenile, or childhood-onset diabetes; type 2 diabetes—as non-insulin-dependent or adult-onset diabetes; LADA diabetes is late autoimmune diabetes of adulthood. Additionally, intermediate conditions such as impaired glucose tolerance and impaired fasting glycemia are recognized as conditions that indicate a high risk of progressing to type 2 diabetes.

In type 1 diabetes, insulin production is absent due to autoimmune destruction of pancreatic beta-cells. There are several markers of this autoimmune destruction, detectable in body fluids and tissues, including islet cell autoantibodies, insulin autoantibodies, glutamic acid decarboxylase autoantibodies, and tyrosine phosphatase ICA512/IA-2 autoantibodies. In type 2 diabetes, comprising 90% of diabetics worldwide, insulin secretion may be inadequate, but peripheral insulin resistance is believed to be the primary defect. Type 2 diabetes is commonly, although not always, associated with obesity, a cause of insulin resistance. It should be further appreciated that the method of the invention is applicable for a subject displaying increased insulin resistance.

Type 2 diabetes is often preceded by pre-diabetes, in which blood glucose levels are higher than normal but not yet high enough to be diagnosed as diabetes. The term pre-diabetes, as used herein, is interchangeable with the terms impaired glucose tolerance or impaired fasting glucose, which are terms that refer to tests used to measure blood glucose levels.

Chronic hyperglycemia in diabetes is associated with multiple, primarily vascular complications affecting microvasculature and/or macrovasculature. These long-term complications include retinopathy (leading to focal blurring, retinal detachment, and partial or total loss of vision), nephropathy (leading to renal failure), neuropathy (leading to pain, numbness, and loss of sensation in limbs, and potentially resulting in foot ulceration and/or amputation), cardiomyopathy (leading to heart failure), and increased risk of infection. Type 2, or noninsulin-dependent diabetes mellitus (NIDDM), is associated with resistance of glucose-utilizing tissues like adipose tissue, muscle, and liver, to the physiological actions of insulin. Chronically elevated blood glucose associated with NIDDM can lead to debilitating complications including nephropathy, often necessitating dialysis or renal transplant; peripheral neuropathy; retinopathy leading to blindness; ulceration and necrosis of the lower limbs, leading to amputation; fatty liver disease, which may progress to cirrhosis; and susceptibility to coronary artery disease and myocardial infarction. By ‘prevent’ it is meant that the risk of developing of diabetes is reduced or the onset of the disease is delayed. By ‘control’ or ‘treat’ it is meant that the risk of developing associated complications is reduced and/or the onset of such complications is delayed.

Diabetic conditions that are subject to treatment with GC or CE or their combinations or their combinations with other drugs, and with insulin, according to the methods of the present invention can be diagnosed or monitored using any of a number of assays known in the field. Examples of assays for diagnosing or categorizing an individual as diabetic or pre-diabetic or monitoring said individual include, but are not limited to, a glycosylated hemoglobin (HbA1c) test, a connecting peptide (C-peptide) test, a fasting plasma glucose (FPG) test, an oral glucose tolerance test (OGTT), and a casual plasma glucose test.

HbA1c is a biomarker that measures the amount of glycosylated hemoglobin in the blood. HbA1c designates a stable minor glycated sub fraction of hemoglobin. It is a reflection of the mean blood glucose levels during the last 6-8 weeks, and is expressed in percent (%) of total hemoglobin. Alternatively, diabetes or pre-diabetes can be diagnosed by measuring blood glucose levels using any of several known tests in the field, including a fasting plasma glucose test or an oral glucose tolerance test. Using the fasting plasma glucose (FPG) test, a patient is classified as diabetic and is subject to treatment according to the methods of the present invention if the patient has a threshold FPG greater than 125 mg/dl, and a patient is classified as pre-diabetic and is subject to treatment according to the methods of the present invention if the patient has a threshold FPG greater than 100 mg/dl but less than or equal to 125 mg/dl. Using the oral glucose tolerance test (OGTT), a patient is classified as diabetic and is subject to treatment according to the methods of the present invention if the patient has a threshold 2-hour OGTT glucose level greater than 200 mg/dl. A patient is classified as pre-diabetic and is subject to treatment according to the methods of the present invention if the patient has a threshold 2-hour OGTT glucose level greater than 140 mg/dl but less than 200 mg/dl.

C-peptide, produced from proinsulin molecules, is secreted from islet cells into the bloodstream in equimolar proportion as insulin, and is used a biomarker for beta-cell function and insulin secretion. A fasting C-peptide measurement greater than 2.0 ng/ml is indicative of high levels of insulin, while a fasting C-peptide measurement less than 0.5 ng/ml indicates insufficient insulin production.

A subject who has been classified as having a diabetic condition, and who is subject to treatment with the combined GC and C:E compositions according to the methods of the present invention, may be monitored for efficacy of treatment by measuring any of the biomarkers and/or blood glucose indicators described herein, including but not limited to, glycosylated hemoglobin levels, C-peptide levels, fasting plasma glucose levels, and oral glucose tolerance test (OGTT) levels. For the biomarkers and/or blood glucose indicators described herein, efficacy of treatment can determined by quantitating the level of a biomarker or blood glucose indicator in a sample from a subject and determining whether the level of the biomarker or blood glucose indicator has reached or is approaching a threshold level. In some embodiments, a threshold level may correspond to a level of biomarker or blood glucose indicator that is a normal (i.e. non-diabetic) value according to standards known in the art, or a threshold level may correspond to a level of biomarker or blood glucose indicator that is a pre-diabetic or diabetic value according to standards known in the art.

In some embodiments, efficacy of treatment is determined by taking a first measurement of one or more of the biomarkers and/or blood glucose indicators in a subject prior to the start of treatment, and comparing the first measurement with secondary measurements of the same biomarker and/or blood glucose indicator in the subject at one or more time points after the onset of treatment, wherein a second measurement that has reached or exceeded a threshold value (either above or below, depending on the biomarker being measured), or is closer to the threshold value than the first measurement is to the threshold value, indicates that the treatment is efficacious.

Alternatively or additionally, efficacy of treatment may be monitored by determining whether there has been an amelioration of the secondary conditions and symptoms that are associated with the diabetic condition. For example, a subject being treated by the methods of the present invention can be monitored for improvement or reduction in symptoms of retinopathy (e.g., improvement in vision), nephropathy (e.g., improvement in kidney structure or function), neuropathy (e.g., improvement in nerve function), and/or cardiovascular disease (e.g., decreased blood pressure or lower lipid levels).

According to another embodiment, the composition of the invention may further lead to a significant reduction in pancreatic hyperplasia and hepatic fat accumulation.

Still further, according to another embodiment, the combined composition of the invention downregulates or later the function macrophages while increasing foxp3+ or any other type of regulatory T cells in fat tissue or in the body, suppresses inflammatory cytokine production by adipocytes and clearly leads to a marked decrease of inflammatory cell infiltration to fat tissue of a treated subject, specifically, a subject suffering from an immune-related disorder.

More particularly, the combined composition of the invention is intended for the treatment of dyslipoproteinemia, which may include hypertriglyceridemia, hypercholesterolemia and low HDL-cholesterol, obesity, NIDDM (non-insulin dependent diabetes mellitus), IGT (impaired glucose tolerance), blood coagulability, blood fibronolysis defects and hypertension.

According to certain embodiments, the immunomodulatory combined composition of the invention is especially advantageous for the treatment of type 1 diabetes or diabetes mellitus, thereby preventing or reducing acute complications (e.g. hypoglycemia, ketoacidosis or nonketotic hyperosmolar coma) as well as long-term complications (e.g. cardiovascular disease, chronic renal failure, retinal damage or blindness, nerve damage and microvascular damage, which may cause impotence, poor healing wounds particularly of the feet potentially leading to gangrene and amputation).

According to some embodiments of some aspects of the present invention, the compositions of the present invention comprising each of the compounds above can be used to prevent, treat and control liver diseases and disorders including hepatitis, cirrhosis, non-alcoholic steatohepatitis (NASH) (also known as non-alcoholic fatty liver disease-NAFLD), hepatotoxicity and chronic liver disease. In general, the terms ‘prevent’, ‘control’ and ‘treat’ encompass the prevention of the development of a disease or a symptom from a patient who may have a predisposition of the disease or the symptom but has yet been diagnosed to have the disease or the symptom; the inhibition of the symptoms of a disease, namely, inhibition or retardation of the progression thereof; and the alleviation of the symptoms of a disease, namely, regression of the disease or the symptoms, or inversion of the progression of the symptoms.

In further embodiments, the method of the invention may optionally further comprises the concurrent or parallel administration of an additional therapeutic agent. In some specific embodiments, such additional therapeutic agent may be any one of insulin, N-acetyl cysteine (NAC), thiamine (vitamin B1), a benzodiazepine, any gut hormone as described above, or any combination thereof.

In some embodiments, the invention provides methods for treating, preventing, ameliorating, reducing or delaying the onset of an immune-related disorder.

Immune mediated conditions are a result of dysbalance of the immune system leading to inflammatory states and autoimmune diseases.

Immune therapy involves the exposure of components of the immune system to various elements (cytokines, disease associated antigens and natural metabolites) to combat disease processes in which a dysregulated immune response is thought to play a role Immune dysregulation is thought to play a major part in the pathogenesis or disease course of a great number of disease processes, including various neoplastic, inflammatory, autoimmune, infectious and genetic entities.

These disorders can be perceived as a dysbalance between pro-inflammatory (Th1) and anti-inflammatory (Th2) cytokines, or any dysbalance of cells that control the immune system whether being a regulatory cell of any kind, antigen presenting cells, or any cells capable of altering the immune system. The way the immune system responds to foreign and self antigens, is the result of a balance between the two subtypes of responses. Experimental autoimmune diseases in humans can be perceived as a dysbalance between pro-inflammatory and anti-inflammatory cytokines, or a dysbalance between cells or cytokines or chemokines.

In the past few years it has been become increasingly clear that T cells capable of actively suppressing immune responses are thought to be in part responsible for the maintenance of peripheral self tolerance. In healthy rodents and humans, there are different types of cells which are able to exert such suppressive function in vitro and in vivo Immunoregulatory cytokines such as IL-10 or TGF-β may be critical for the suppressive effect of these cells. Regulatory T cells have potential role in human autoimmune or chronic inflammatory diseases and can be used for diagnostic or therapeutic purposes.

In more specific embodiments, such immune-related disorder may be any one of an inflammatory disorder, an autoimmune disorder, an infectious disease and a proliferative disorder.

In yet another embodiment, the pharmaceutical composition and methods of the invention may be used for the treatment of an autoimmune disorder. Examples of autoimmune disorders include, but are not limited to, Asthma, Primary sclerosing cholangitis, Alopecia Areata, Lupus, Anlcylosing Spondylitis, Meniere's Disease, Antiphospholipid Syndrome, Mixed Connective Tissue Disease, Autoimmune Addison's Disease, Multiple Sclerosis, Autoimmune Hemolytic Anemia, Myasthenia Gravis, Autoimmune Hepatitis, Pemphigus Vulgaris, Behcet's Disease, Pernicious Anemia, Bullous Pemphigoid, Polyarthritis Nodosa, Cardiomyopathy, Polychondritis, Celiac Sprue-Dermatitis, Polyglandular Syndromes, Chronic Fatigue Syndrome (CFIDS), Polymyalgia Rheumatica, Chronic Inflammatory Demyelinating, Polymyositis and Dermatomyositis, Chronic Inflammatory Polyneuropathy, Primary Agammaglobulinemia, Churg-Strauss Syndrome, Primary Biliary Cirrhosis, Cicatricial Pemphigoid, Psoriasis, CREST Syndrome, Raynaud's Phenomenon, Cold Agglutinin Disease, Reiter's Syndrome, Crohn's Disease, Rheumatic Fever, Discoid Lupus, Rheumatoid Arthritis, Essential Mixed, Cryoglobulinemia Sarcoidosis, Fibromyalgia, Scleroderma, Grave's Disease, Sjogren's Syndrome, Guillain-Barre, Stiff-Man Syndrome, Hashimoto's Thyroiditis, Takayasu Arteritis, Idiopathic Pulmonary Fibrosis, Temporal Arteritis/Giant Cell Arteritis, Idiopathic Thrombocytopenia Purpura (ITP), Ulcerative Colitis, IgA Nephropathy, Uveitis, Insulin Dependent Diabetes (Type I), Vasculitis, Lichen Planus, and Vitiligo. The oral combined beta-glycolipid and Cremophore EL compositions described herein can be administered to a subject to treat or prevent disorders associated with an abnormal or unwanted immune response associated with cell, tissue or organ transplantation, e.g., renal, hepatic, and cardiac transplantation, e.g., graft versus host disease (GVHD), or to prevent allograft rejection.

According to a specifically preferred embodiment, an autoimmune disease treated by the composition of the invention may be any one of rheumatoid arthritis, type 1 diabetes, type 2 diabetes, artherosclerosis, asthma, acute and chronic graft versus host disease, systemic lupus erythmatosus, scleroderma, multiple sclerosis, inflammatory bowel disease, psoriasis, uvietis, thyroiditis and immune mediated hepatitis.

According to another embodiment, the combined composition of the invention may be used for the treatment of MS. Multiple Sclerosis (MS) is typically characterized clinically by recurrent or chronically progressive necrologic dysfunction, caused by lesions in the CNS. Pathologically, the lesions include multiple areas of demyelination affecting the brain, optic nerves, and spinal cord. The underlying etiology is uncertain, but MS is widely believed to be at least partly an autoimmune or immune-mediated disease.

Thus, the invention includes compositions and methods of treating, delaying or preventing the onset of MS, by orally or mucosally administering the combined beta-glycolipid and Cremophore EL. Included are methods wherein a subject who has or is at risk of having MS is orally administered combined beta-glycolipid and Cremophore EL.

According to another preferred embodiment, the combined composition of the invention may be used for the treatment of any inflammatory arthritis. In specific embodiments, the compositions and methods of the invention may be applicable for treating Rheumatoid arthritis (RA). RA is the most common chronic inflammatory arthritis and affects about 1% of adults, it is two to three times more prevalent in women than in men. RA may begin as early as infancy, but onset typically occurs in the fifth or sixth decade.

Diagnosis may be made according to the American Rheumatism Association Criteria for the so Classification of Rheumatoid Arthritis. A therapeutically effective amount will cause an improvement in one or more of the following: the number of inflamed joints, the extent of swelling, and the range of joint motion. Laboratory measurements (e.g., ESR and hematocrit value) and assessments of subjective features (e.g., pain and morning stiffness) can also be made. The invention also includes methods of treating autoimmune arthritis, e.g., RA, in a subject by administering to the subject a therapeutically effective amount of combined composition of the invention comprising beta-glycolipid and Cremophore EL.

The combined compositions of the invention described herein can also be used to treat or prevent graft rejection in a transplant recipient. For example, the compositions can be used in a wide variety of tissue and organ transplant procedures, e.g., the compositions can be used to induce central tolerance in a recipient of a graft of cells, e. g., stem cells such as bone marrow and/or of a tissue or organ such as pancreatic islets, liver, kidney, heart, lung, skin, muscle, neuronal tissue, stomach, and intestines. Thus, the new methods can be applied in treatments of diseases or conditions that entail cell, tissue or organ transplantation (e.g., liver transplantation to treat hypercholesterolemia, transplantation of muscle cells to treat muscular dystrophy, or transplantation of neuronal tissue to treat Huntington's disease or Parkinson's disease).

According to another embodiment, the combined composition of the invention may modulate the T cells or other cells balance towards a suppressing response in a subject suffering from IBD. Therefore, according to one embodiment, the composition of the invention is intended for treating IBD. Inflammatory bowel diseases (IBD) are common gastrointestinal disorders, that can be perceived as being the result of a dysbalance between pro-inflammatory and anti-inflammatory subtypes of immune responses.

Patients with IBD have antibodies against components of colon cells and several different bacterial antigens. These antigens gain access to the immune system as a consequence of epithelial damage. Abnormalities of T cell-mediated immunity, including coetaneous anergy and diminished responsiveness to T cell stimuli, have also been described in these patients. In addition, changes in mucosal cell mediated immunity were identified, including increased concentrations of mucosal IgG cells and changes in T cells subsets, suggesting antigen stimulation. Exposure of target antigens after infectious, immune, or toxic damage, leads to activation of mucosal immune cells resulting in cytokines that lead to mucosal inflammatory response. Secretion of pro-inflammatory cytokines such as IFNγ, contributes to an increase in mucosal permeability, and has been described in animal models of IBD.

In yet another preferred embodiment, the combined composition of the invention may be used for the treatment of atherosclerosis. Atherosclerosis is a slowly progressive disease characterized by the accumulation of cholesterol within the arterial wall. The atherosclerotic process begins when LDL-C becomes trapped within the vascular wall. Oxidation of the LDL-C results in the bonding of monocytes to the endothelial cells lining the vessel wall. These monocytes are activated and migrate into the endothelial space where they are transformed into macrophages, leading to further oxidation of LDL-C. The oxidized LDL-C is taken up through the scavenger receptor on the macrophage leading the formation of foam cells. A fibrous cap is generated through the proliferation and migration of arterial smooth muscle cells, thus creating an atherosclerotic plaque. Lipids depositing in atherosclerotic legions are derived primarily from plasma apo B containing lipoproteins. These include chylomicrons, LDL-C, IDL, and VLDL. This accumulation forms bulky plaques that inhibit the flow of blood until a clot eventually forms, obstructing an artery and causing a heart attack or stroke.

Thus, in another specific embodiment, the combined composition of the invention is intended for the treatment of a malignancy. In cancerous situations, modulation of the T cell balance may be in the direction of inducing a pro-inflammatory response or in augmenting the anti-tumor associated antigens immunity. As used herein to describe the present invention, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. In general, the compositions of the present invention may be used in the treatment of non-solid and solid tumors.

Malignancy, as contemplated in the present invention may be selected from the group consisting of carcinomas, melanomas, lymphomas, myeloma, leukemia and sarcomas. Malignancies that may find utility in the present invention can comprise but are not limited to hematological malignancies (including leukemia, lymphoma and myeloproliferative disorders), hypoplastic and aplastic anemia (both virally induced and idiopathic), myelodysplastic syndromes, all types of paraneoplastic syndromes (both immune mediated and idiopathic) and solid tumors (including lung, liver, breast, colon, prostate GI tract, pancreas and Karposi). More particularly, the malignant disorder may be hepaotcellular carcinoma, colon cancer, melanoma, myeloma, acute or chronic leukemia.

It should be noted that the immuno-modulatory composition of the invention may be applicable for treating infectious diseases caused by bacterial infections, viral infections, fungal infections, or parasitic infections. More specifically, the viral infection may be caused by any one of HBV, HCV or HIV.

In some specific embodiments, the method of the invention may be suitable for treating an immune-related disorder, for example, hepatitis.

In some embodiments, the method of the invention may further comprise the concurrent or parallel administration of at least one additional therapeutic agent. In certain embodiments such agent is any one of insulin, antibodies directed to inflammatory cytokine, or antibodies such as anti TNF antibodies including humanized antibodies, statins, analgesics, chemotherapeutic agents and antibiotics.

Still further embodiments relate to the method of the invention for treating liver damage in a subject in need thereof. More specifically, such method uses a composition comprises a therapeutically effective amount of a natural or synthetic beta-glycolipid and polyethoxylated castor oil or any derivative thereof, or any combination thereof.

In more specific embodiments, such subject may be a subject suffering from a liver disease, that may be any one of viral, alcoholic or autoimmune hepatitis, alcoholic or autoimmune cirrhosis, alcoholic fatty liver disease, non alcoholic fatty liver disease (NAFLD), any type of liver steatosis, for example, due to other disease such as Wilson's disease or alpha 1 anti trypsin deficiency, alcoholic or nonalcoholic steatohepatits (NASH), hepatocellular carcinoma, drug-induced liver disease and pediatric liver disease and any type of metabolic liver disease, for example, glycogen storage disease

The terms “liver disease” or “liver damage” as used herein apply to many diseases and disorders that cause the liver to function improperly or to cease functioning, and this loss of liver function is indicative of liver disease. Thus, liver function tests are frequently used to diagnose liver disease. Examples of such tests include, but are not limited to, the following;
Assays to determine the levels of serum enzymes such as lactate dehydrogenase (LDH), alkaline phosphatase (ALP), aspartate aminotransferase (AST), and alanine aminotransferase (ALT), where an increase in enzyme levels indicates liver disease. One of skill in the art will reasonably understand that these enzyme assays indicate only that the liver has been damaged. They do not assess the liver's ability to function. Other tests can be used to assay a liver's ability to function.
Assays to determine serum bilirubin levels. Serum bilirubin levels are reported as total bilirubin and direct bilirubin. Normal values of total serum bilirubin are 0.1-1.0 mgdl (e.g., about 2-18 mmol/L). Normal values of direct bilirubin are 0.0-0.2 mg/dl (0-4 mmol/L). Increases in serum bilirubin are indicative of liver disease.
Assays to determine serum protein levels, for example, albumin and the globulins (e.g., alpha, beta, gamma). Normal values for total serum proteins are 6.0-8.0 g/dl (60-80 g/L). A decrease in serum albumin is indicative of liver disease. An increase in globulin is indicative of liver disease.

Other tests include prothrombin time, international normalized ratio, activated clotting time (ACT), partial thromboplastin time (PTT), prothrombin consumption time (PCT), fibrinogen, coagulation factors; alpha-fetoprotein, and alpha-fetoprotein-L3 (percent).

As shown in Example 4, the combined composition of the invention has been shown as significantly ameliorating conA induced hepatitis thereby establishing the feasibility of using the composition of the invention for treating any liver damage One clinically important type of liver disease is hepatitis. Hepatitis is an inflammation of the liver that can be caused by viruses (e.g., hepatitis virus A, B and C (HAV, HBV, and HCV, respectively), chemicals, drugs, alcohol, inherited diseases, or the patient's own immune system (autoimmune hepatitis). This inflammation can be acute and resolve within a few weeks to months, or chronic, and persist over many years. Chronic hepatitis can persist for decades before causing significant symptoms, such as cirrhosis (scarring and loss of function), liver cancer, or death. Other important examples of the different diseases and disorders encompassed by the term “liver disease” and suitable for treatment or prevention or control using the compositions and methods of the present invention include, but are not limited to amebic liver abscess, biliary atresia, fibrosis, cirrhosis, coccidioidomycosis, delta agent, hepatocellular carcinoma (HCC), alcoholic liver disease, primary biliary cirrhosis, pyogenic liver abscess, Reye's syndrome, sclerosing cholangitis, and Wilson's disease. In some embodiments, the compositions and methods described herein are suitable for the treatment of liver disease characterized by the loss or damage of parenchymal liver cells. In some aspects, the etiology of this can be a local or systemic inflammatory response.

Liver failure occurs when large parts of the liver become damaged and the liver is no longer able to perform its normal physiological function. In some aspects, liver failure can be diagnosed using the above described assays of liver function or by a subject's symptoms. Symptoms that are associated with liver failure include, for example, one or more of the following, nausea, loss of appetite, fatigue, diarrhea, jaundice, abnormal/excessive bleeding (e.g., coagulopathy), swollen abdomen, mental disorientation or confusion (e.g., hepatic encephalopathy), sleepiness, and coma.

Chronic liver failure occurs over months to years and is most commonly caused by viruses (e.g., HBV and HCV), long-term/excessive alcohol consumption, cirrhosis, hemochromatosis, and malnutrition. Acute liver failure is the appearance of severe complications after the first signs of liver disease (e.g., jaundice) and includes a number of conditions, all of which involve severe hepatocyte injury or necrosis. In some embodiments, the compositions and methods described herein are particularly suitable for the treatment of hyperacute, acute, and subacute liver failure, fulminant hepatic failure and late onset fulminant hepatic failure, all of which are referred to herein as “acute liver failure.” Common causes for acute liver failure include, for example, viral hepatitis, exposure to certain drugs and toxins (e.g., fluorinated hydrocarbons (e.g., trichloroethylene and tetrachloroethane), amanita phalloides (e.g., commonly found in the “death-cap mushroom”), acetaminophen (paracetamol), halothanes, sulfonamides, henytoins), cardiac-related hepatic ischemia (e.g., myocardial infarction, cardiac arrest, cardiomyopathy, and pulmonary embolism), renal failure, occlusion of hepatic venous outflow (e.g., Budd-Chiari syndrome), Wilson's disease, acute fatty liver of pregnancy, amebic abscesses, and disseminated tuberculosis.

The term hepatitis is used to describe a liver condition which implies injury to the liver characterized by the presence of inflammatory cells in the tissue of the organ. The condition can be self-limiting, healing on its own, or can progress to scarring of the liver. Hepatitis is acute when it lasts less than six months and chronic when it persists longer than six months. A group of viruses known as the hepatitis viruses cause most cases of liver damage worldwide. Hepatitis can also be due to toxins (notably alcohol), other infections or from autoimmune process. Hepatitis includes hepatitis from viral infections, including Hepatitis A through E (A, B, C, D and E—more than 95% of viral cause), Herpes simplex, Cytomegalovirus, Epstein-Barr virus, yellow fever virus, adenoviruses; non-viral infections, including toxoplasma, Leptospira, Q fever, rocky mountain spotted fever, alcohol, toxins, including amanita toxin in mushrooms, carbon tetrachloride, asafetida, among others, drugs, including paracetamol, amoxycillin, antituberculosis medicines, minocycline and numerous others as described herein; ischemic hepatitis (circulatory insufficiency); pregnancy; autoimmune conditions, including Systemic Lupus Erythematosus (SLE); and non-alcoholic steatohepatitis.

Sterile inflammation is used to describe inflammation of the liver which is triggered by intracellular molecules released from dying cells that have lost integrity of their plasma membrane. This inflammation occurs in the absence of causative agents such as viruses or bacteria and alcohol. A number of intracellular molecules have been identified that can stimulate other cells to produce proinflammatory cytokines and chemokines. Such proinflammatory cellular molecules are thought to function by engaging receptors on cytokine-producing cells. If left untreated, sterile inflammation may progress to non-alcoholic fatty liver disease (NAFLD), non-alcoholic steatohepatitis (NASH) or cyrrhosis.

Non-alcoholic steatohepatitis or NASH is a condition of the liver in which inflammation is caused by a buildup of fat in the liver. NASH is part of a group of liver diseases, known as nonalcoholic fatty liver disease, in which fat builds up in the liver and sometimes causes liver damage that gets worse over time (progressive liver damage). “Non-alcoholic fatty liver disease” (NAFLD) is fatty inflammation of the liver which is not due to excessive alcohol use. It is related to insulin resistance and the metabolic syndrome, obesity, high cholesterol and triglycerides, and diabetes, and may respond to treatments originally developed for other insulin resistant states (e.g. diabetes mellitus type 2), such as weight loss, metformin and thiazolidinediones. Non-alcoholic steatohepatitis (NASH) is the most extreme form of NAFLD, which is regarded as a major cause of cirrhosis of the liver of unknown cause.

Other factors that have been known to contribute to NASH include: surgery that shorten the intestines, the stomach, or both, such as jejunal bypass operation or biliopancreatic diversion; prolonged use of feeding tube or other method of receiving nutrition; certain drugs, including amiodarone, glucocorticoids, synthetic estrogens, and tamoxifen.

NASH is a condition that may get worse over time (called a progressive condition) and can cause scarring (fibrosis) of the liver, which leads to cirrhosis. “Cirrhosis” describes a condition in which liver cells have been replaced by scar tissue. The term “cirrhosis of the liver” or “cirrhosis” is used to describe a chronic liver disease characterized by replacement of liver tissue by fibrous scar tissue as well as regenerative nodules, leading to progressive loss of liver function. Cirrhosis is most commonly caused by fatty liver disease, including NASH, as well as alcoholism and hepatitis B and C, but also may be of unknown cause. Potentially life-threatening complications of cirrhosis are hepatic encephalopathy (confusion and coma) and bleeding from esophageal varices. Cirrhosis has historically been thought to be generally irreversible once it occurs, and historical treatment focused on preventing progression and complications. In advanced stages of cirrhosis, the only option is a liver transplant.

Each of the compounds above, specifically in the combined compositions and methods of the present invention can be used to treat, prevent or control chemical liver trauma and hepatotoxicity. Also chemical trauma or acute chemical trauma to the liver refers to serious injury which occurs to a patient over a short duration as a consequence of chemical toxicity, including drug-induced toxicity or trauma. Drug-induced acute liver trauma, including acetaminophen-induced acute liver trauma, is acute liver injury which occurs as a result or consequence of exposure to a drug (e.g., drug overdose), especially acetaminophen toxicity. Compounds according to the present invention are useful for reducing the injury to the liver which occurs from physical and chemical trauma, especially including drug-induced (drug overdose) and acetaminophen-induced acute liver trauma.

Hepatotoxocity is chemical liver trauma resulting from a hepatotoxic agent, or hepatotoxicity-inducing bioactive agent. The terms “hepatotoxic agent” and “a hepatotoxicity inducing bioactive agent” are used synonymously in context to describe compounds which often produce hepatotoxicity in patients administered such agents. Examples of hepatoxicity agents include, for example, anaesthetic agents, antiviral agents, anti-retroviral agents (nucleoside reverse transcriptase inhibitors and non-nucleoside reverse transcriptase inhibitors), especially anti-HIV agents, anticancer agents, organ transplant drugs (cyclosporin, tacrolimus, OKT3), antimicrobial agents (anti-TB, anti-fungal, antibiotics), anti-diabetes drugs, vitamin A derivatives, steroidal agents, especially including oral contraceptives, anabolic steroids, androgens, non-steroidal anti-inflammatory agents, anti-depressants (especially tricyclic antidepressants) glucocorticoids, natural products and herbal and alternative remedies, especially including St. John's wort. Hepatotoxicity may manifest as triglyceride accumulation which leads to either small droplet (microvesicular) or large droplet (macrovesicular) fatty liver. There is a separate type of steatosis where phospholipid accumulation leads to a pattern similar to the diseases with inherited phospholipid metabolism defects (e.g. Tay-Sachs disease).

It must be understood that the combined compositions and methods of the invention are particularly applicable for treating any of the hepatic disorders described herein above.

In certain embodiments, the method of the invention may optionally further comprises the concurrent or parallel administration of at least one additional therapeutic agent. More specifically, such additional therapeutic agent may be any one of insulin, NAC, vitamin B1, a benzodiazepine, an anti-viral or anti-inflammatory drug, a chemotherapeutic agent and any gut hormone.

In further alternative embodiments, the method of the invention may be applicable for treating, preventing, ameliorating, reducing or delaying the onset of acute or chronic toxic effect of a drug.

In certain embodiments, such drug may be an analgesic or an antipyretic drug.

It is understood that the methods of the invention involve administering the combined compositions of the invention, specifically, compositions comprising beta glucosylceramide (GC) and Cremophore EL, or alternatively, GC with PEG or GC with cyclo dextrin or any derivative/s thereof. There are numerous administration routes that may be used. In some embodiments, the administration is at least one of oral, mucosal, nasal, transdermal, pulmonary, buccal or sublingual administration, or any combinations thereof. Other administration modes are also applicable, for example, subcutaneous, rectal, or parenteral (including intramuscular, intraperitoneal (IP), intravenous (IV) and intradermal) administration.

An amount adequate to accomplish this is defined as a “therapeutically effective dose.” Amounts effective for this use will depend upon the severity of the condition and the general state of the patient's own immune system, but generally range from about 0.001 to about 1000 mg/Kg of the glucosylceramide and of the Cremophore EL of the invention. Specifically, the glucosylceramide and of the Cremophore EL, in dosages of from 0.0001 to 5000 mg and 0.01 to 2.5, specifically, 0.001, 0.002, 0.003, 0.004, 0.005, 0.01, 0.02, 0.03, 0.04, 0.05, 0.06, 0.07, 0.08, 0.09, 0.1, 0.2, 0.3 0.4, 0.5, 0.6, 0.7, 0.8, 0.9, 1, 1.5, 2, 2.5, 3, 3.5, 4, 4.5 and 5 mg. More specifically, about 0.005 to 2.5 and most specifically, a low dose of 0.0.00001 mg or a high dose of 10000000 mg the glucosylceramide and Cremophore EL per Kg of body weight being more commonly used. Single or multiple administrations on a daily, weekly or monthly schedule can be carried out with dose levels and pattern being selected by the treating physician.

In some specific embodiments, where the composition of the invention, specifically, the combined GC and CE composition is an add-on composition to SSB, the effective amount may range between about 0.1 ng GC to about 1000 gr GC in CE added-on 500 ml of SSB, More specifically, about 0.1, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 mg or more, specifically, 150, 200, 250, 300, 350, 400, 450, 500, 550, 600, 650, 700, 750, 800, 850, 900, 950 or 1000 mg GC in CE 0.0001 ng to 1000 gr added on 500 ml of SSB. In yet more specific embodiments, the invention encompasses the addition of about 28 mg GC in CE in 500 ml of SSB.

As discussed above, the invention provides different methods of treating, ameliorating preventing or delaying the onset of hepatic or any immune-related disorders in a subject in need. As used herein in the specification and in the claims section below, the term “treat” or “treating” and their derivatives includes substantially inhibiting, slowing or reversing the progression of a condition, substantially ameliorating symptoms of a condition or substantially preventing the appearance of symptoms of a condition, said condition is any one of an immune-related disorder and a hepatic disorder in a subject in need thereof.

The term “prevent” and all variations of this term is intended to mean the countering in advance of pathologic symptoms or a pathologic process progress. In this case it is understood that the composition is applied prior to the observation of clinical symptoms.

The terms “ameliorate” and “amelioration” relate to the improvement in the treated subject condition brought about by the compositions and methods according to the invention, wherein said improvement may be manifested in the forms of inhibition of pathologic processes associated with any one of an immune-related disorder and a hepatic disorder, a significant reduction in their magnitude, or an improvement in a diseased subject physiological state.

The term “inhibit” and all variations of this term is intended to encompass the restriction or prohibition of the progress and exacerbation of pathologic symptoms or a pathologic process progress, said pathologic process symptoms or process are associated with.

The term “eliminate” relates to the substantial eradication or removal of the pathologic symptoms and possibly pathologic etiology, optionally, according to the methods of the invention described below.

The terms “delay”, “delaying the onset”, “retard” and all variations thereof are intended to encompass the slowing of the progress and/or exacerbation of an immune-related disorder or a hepatic disorder and their symptoms slowing their progress, further exacerbation or development, so as to appear later than in the absence of the treatment according to the invention.

By “subject in need” or “patient” it is meant any mammal who may be affected by the above-mentioned conditions, and to whom the treatment and diagnosis methods herein described is desired, including human, bovine, equine, canine, murine and feline subjects. Preferably, the patient is a human. Administering of the composition according to the method of the invention to the patient includes both self-administration and administration to the patient by another person.

The invention further encompasses the use of the composition and methods of the invention for treating any condition related to the conditions described above. It is understood that the interchangeably used terms “associated” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology described herein.

In a further aspect, the invention provides a pharmaceutical composition for use in a method for treating liver damage in a subject in need thereof. More specifically, such composition may comprise as an active ingredient a therapeutically effective amount of a polyethoxylated castor oil or any derivative or a combination thereof, and optionally further comprising a pharmaceutically acceptable carrier.

Still further, the invention provides a soft or an alcoholic beverage comprising at least one polyethoxylated castor oil or any derivative and at least one a natural or synthetic beta-glycolipid.

The invention further provides a combined composition comprising as an active ingredient at least one natural or synthetic beta-glycolipid and at least one polyethoxylated castor oil or any derivative/s thereof.

It should be appreciated that the invention provides in a further aspect thereof, at least one high sugar or alcoholic beverage, for example, SSB, chocolate milk and the like, that further comprise a combination of GC and polyethoxylated castor oil (CE) or any derivative thereof.

Still further, the invention provides in some embodiments thereof at least one high sugar or alcoholic beverage, for example, SSB, chocolate milk and the like, that further comprise a combination of GC and PEG or any derivative thereof.

In further embodiments, the invention provides at least one high sugar or alcoholic beverage, for example, SSB, chocolate milk and the like, that further comprise a combination of GC and cyclo dextrin or any derivative thereof.

The term ‘Sugar Sweetened Beverages’ (SSBs) as meant herein refers to beverages with high sugar content and/or those associated with higher caloric intakes. The 2010 Dietary Guidelines for Americans define SSBs as “liquids that are sweetened with various forms of sugars that add calories. These beverages include, but are not limited to, soda, fruit drinks, and sports and energy drinks”. In the National Health and Nutrition Examination Survey (NHANES) 2005-2008, about half of Americans drank SSBs on any given day. SSB intake in adults is associated with obesity, type 2 diabetes, and increased risk for cardiovascular disease, nonalcoholic fatty liver disease, kidney disease, gout and decreased diet quality.

The present invention also relates to sweetened soft drinks (also soda, pop, soda pop, coke, soda pop, fizzy drink, seltzer, mineral, lolly water or carbonated beverage) that is a beverage that typically contains carbonated water, a sweetener and a flavoring. The sweetener may be sugar, high-fructose corn syrup, fruit juice, sugar substitutes (in the case of diet drinks) or some combination of these. An average can of sugared soda or juice contains about 10 to 12 teaspoons of sugar. Soft drinks may also contain caffeine, colorings, preservatives and other ingredients.

Among the popular SSBs, of particular relevance to the present context is Coca Cola (or coke), which for the purpose of the present invention refers to any carbonated soft drink flavored with coca leaves, cola nuts, caramel, etc, commercially available by other brand names.

Of further relevance to the present invention are milked beverages containing added sugars. Although not classified as SSBs, most flavored milks contain at least double the sugar of plain milk Flavored milk is cow's milk with added flavoring and sweetener, which is available in flavors such as chocolate, strawberry and vanilla flavors in low-fat and fat-free varieties. Most chocolate milks are sweetened with sugar or high fructose corn syrup.

In specific embodiments, the present invention applies to syrups and beverages containing sweeteners high in sugar (more than 95% sugar), such as brown sugar (97%), fructose (93%), honey (82%), high-fructose corn-syrup (76%), molasses (75%), agave syrup and maple syrup (68%), pancake syrups (42-68%), and Canadian maple syrup (60%).

In further embodiments, the invention applies to drink powders and drink concentrates high in sugar content (95% sugar), such as lemonade powder (95%), orange breakfast drink (92%), chocolate ,ilk drink (84%), Gatorade mix (81%), melted chocolate drink mix (67%), cocoa mix powder (66%), instant coffee with whitener, reduced-sugar (59%), instant mocha coffee (58%), pink lemonade concentrate (46%), fruit drink (16%), cream soda and energy drink (13%), Cola, root beer and orange drink (11%), and lemon ice tea and lemon-lime soda (10%).

Yet in other embodiments, the present invention applies to foods with high sugar content, such as candies and nougat high in sugar (90% sugar), e.g. hard candies (93%), butterscotch (81%), vanilla fudge (80%), Skittles (76%), chocolate fudge (73%), chocolate coated fondant (71%), jelly beans and low calorie gum drops (70%), Taffy (69%), high vitamin C fruit snacks (68%), After Eight Mints (67%), chewing gum and caramels (66%).

The present invention further applies to foods containing dried fruits high in sugar (up to 80% sugar), such as blueberries, sweetened (68%), currants, dates and sweetened cherries (67%), cranberries, sweetened (65%), pears (62%), raisins (59%), apricots (53%), figs (48%), bananas (47%), peaches (42%), and prunes (38%).

In specific embodiments, the present invention applies to cookies, cakes and pies high in sugar (up to 70% sugar), such as chocolate sandwich cookies (61%), white cake with coconut frosting (57%), soft raisin cookies (48%), fortune cookies & chocolate covered marshmallows (45%), cream-filled wafers & coffee cake (43%), oatmeal cookies & yellow cake, with vanilla frosting (42%), chocolate cake (40%), diet chocolate chip cookies (40%), reduced fat chocolate brownies (39%), sugar cookies (38%), chocolate chip cookies & sponge cake (37%), coconut cream pie & Boston cream pie (36%), doughnuts, glazed (35%), blueberry muffins (33%), reduced-fat pie crust (30%), mince pies (28%), and pecan pie (25%).

The invention further applies to jams, preserves and spreads high in sugar (up to 60% sugar), such as chocolate-hazelnut spread (54%), most jams (49%), apricot jam (43%), diet jam (38%), chunky peanut butter (11%), and smooth peanut butter (10%).

In specific embodiments, the invention applies to cereals high in sugar (up to 56% sugar), such as Marshmallow Froot Loops (50%), Berry Colossal Crunch (44%), Cinnabon (42%), Frosted Rice Crispies (40%), Cocoa Crispies (39%), Frosted Flakes (38%), Cocoa Puffs (37%), Lucky Charms (36%), Golden Grahams (35%), Raisin Bran (34%), Low Fat Fruit Granola and Honey Nut Cheerios (33%), Special K Fruit And Yogurt (32%), Fruit And Nut Muesli (31%), Special K Red Berries (30%).

In specific embodiments, the invention applies to sauces and instant gravies high in sugar (up to 40%), such as cranberry sauce (38%), pickle relish (29%), Hoisin sauce (27%), pork gravy powder (25%), instant beef gravy (24%), peanut sauce (19%), sweet & sour sauce (19%), teriyaki sauce (14%), cocktail sauce (12%), tomato chili sauce (11%), pasta sauces (6-10%), cheese sauce mix, steak sauce and Worcestershire sauce (10%), instant turkey gravy (7%), salsa (4-6%), and Tartar sauce (4%).

In further embodiments, the invention applies to ice creams, frozen yogurts and milk shakes high in sugar (up to 25% sugar), such as chocolate ice cream and light chocolate ice cream (25%), frozen vanilla soft-serve yogurt (24%), light vanilla ice cream (22%), thick chocolate milk shake, vanilla ice cream and fat free vanilla ice cream (21%), 98% fat free chocolate ice cream (20%), chocolate frozen yogurt (19%), chocolate covered ice cream bar and thick vanilla milk shake (18%), non-fat, no sugar frozen yogurt (13%), fat-free, no sugar ice cream (9%).

Of particular relevance to the present invention are fruit canned in syrup high in sugar (up to 55% sugar), such as Maraschino cherries (39%), plums, sour red cherries and strawberries (22%), figs (21%), blueberries, raspberries, apricots & blackberries (20%), grapes & peaches (19%), fruit salad (18%), fruit cocktail & pineapple (17%), pears, sweet cherries (16%), and mandarin segments (15%).

The present invention is further relevant to alcoholic beverages. An alcoholic beverage is any fermented liquor, such as wine, beer, or distilled spirit, that contains ethyl alcohol, or ethanol (CH₃CH₂OH), as an intoxicating agent. In the US, a standard drink contains 0.6 ounces (14.0 grams or 1.2 tablespoons) of pure alcohol. Generally, this amount of pure alcohol is found in 12-ounces of beer (5% alcohol content); 8-ounces of malt liquor (7% alcohol content); 5-ounces of wine (12% alcohol content); 1.5-ounces of 80-proof (40% alcohol content) distilled spirits or liquor (e.g., gin, rum, vodka, whiskey).

It should be appreciated that the invention further encompasses the use of the combined compositions of the invention in healthy people for prevention of liver steatosis or liver disease when exposed to conditions that possibly can induce any type of liver disease.

It should be further noted that the invention provides methods and compositions for prevention of diabetes in patients with pre diabetes.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise. Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

### Experimental Procedures

Animals

Male C57B1/6 mice (11-12 weeks old) were obtained from Harlan Laboratories (Jerusalem, Israel) and maintained in the Animal Core of the Hadassah-Hebrew University Medical School. All mice were administered standard laboratory chow and water ad libitum and kept in a 12-hour light/dark cycle. SSBs (Coca Cola or orange flavored soda) including additives GC and/or CE were orally administered in a volume of 300-350 μl per mouse by gavage. The animal experiments were carried out according to the guidelines of the Hebrew University-Hadassah Institutional Committee for Care and Use of Laboratory Animals and with the committee's approval.

GC and/or CE Add-on to SSBs

Beta-glucosylceramide (GC) solution consisted of 60 mg GC (purchased from Avanti Polar Lipids; Alabaster, Ala., USA) dissolved in 1 ml mixture of 30% Cremophor EL (Sigma, Rehovot, Israel) and ethanol (1:1; CE) or ethanol alone. These served basis for preparation of SSBs containing GC and/or CE, including 1800 μl Coca Cola or other SSB and 400 μl GC in CE or ethanol or CE alone.

Concanavalin A

Concanavalin A solution (ConA; purchased from MP Biomedicals, USA) consisted of 2 mg ConA in 1 ml distilled water. Mice were intravenously (IV) injected with 250 μl ConA solution (0.5 mg/mouse).

Acetaminophen

Acetaminophen powder (APAP; was purchased from Sigma, cat. # A7085), was dissolved in a mixture of 30% Cremophor EL (Sigma, Rehovot, Israel) and ethanol (1:1) in DDW (Di distilled water).

AST and ALT Levels as Parameters of Liver Injury

Mice were tested for serum alanine transaminase (ALT) and aspartate aminotransferase (AST) at 24 hours after acetaminophen administration. Serum AST and ALT levels were measured by an automatic analyzer.

GTT (Glucose Tolerance Test)

Mice undergo a glucose tolerance test on day 60. Glucose is administered orally (1 g per kg). Serum glucose measurements is performed on tail-vein blood every fifteen minutes for three hours. Glucose levels are measured by a standard glucometer.

Statistical Analyses

Glucose serum concentration was calculated as Area Under the Curve (AUC) values at discrete time points (0, 15, 30, 60 and 180 min) after GC and/or CE administration. AUC was used as an estimate of a total glucose exposure over time under various experimental conditions, i.e. GC and/or CE administration.

Comparison of two independent groups was performed using the Student's t test. The association between two variables was assessed by calculating the Pearson and the Spearman correlation coefficients. All tests applied were two-tailed, and a p value of 0.05 or less was considered statistically significant.

### Example 1

### GC and CE Add-On to SSBs Attenuate Increase of Serum Sugar Levels

To evaluate the ability of GC to control blood sugar levels during consumption of a high glucose beverage, the inventors examined the effect of GC alone and of GC with CE combination, on mice consuming various high sugar soft drinks. Table 1 shows the experimental groups of mice, including group A receiving orally Coca Cola +GC with CE (350 μl/mouse by gavage) and group B receiving Coca Cola (300 μl/mouse by gavage), using experimental procedures detailed above. Glucose levels were measured at 0, 15, and 30 min after Coca Cola administration.

TABLE 1
The effect of GC + C:E on blood sugar levels
after consumption of Coca Cola

| Group | Treatment | AUC 30 minutes |
|---|---|---|
| A | Coca Cola + GC | 4381.25 ± 320 |
| N = 6 | | |
| B | Coca Cola alone | 4785 ± 208 |
| N = 6 | | |

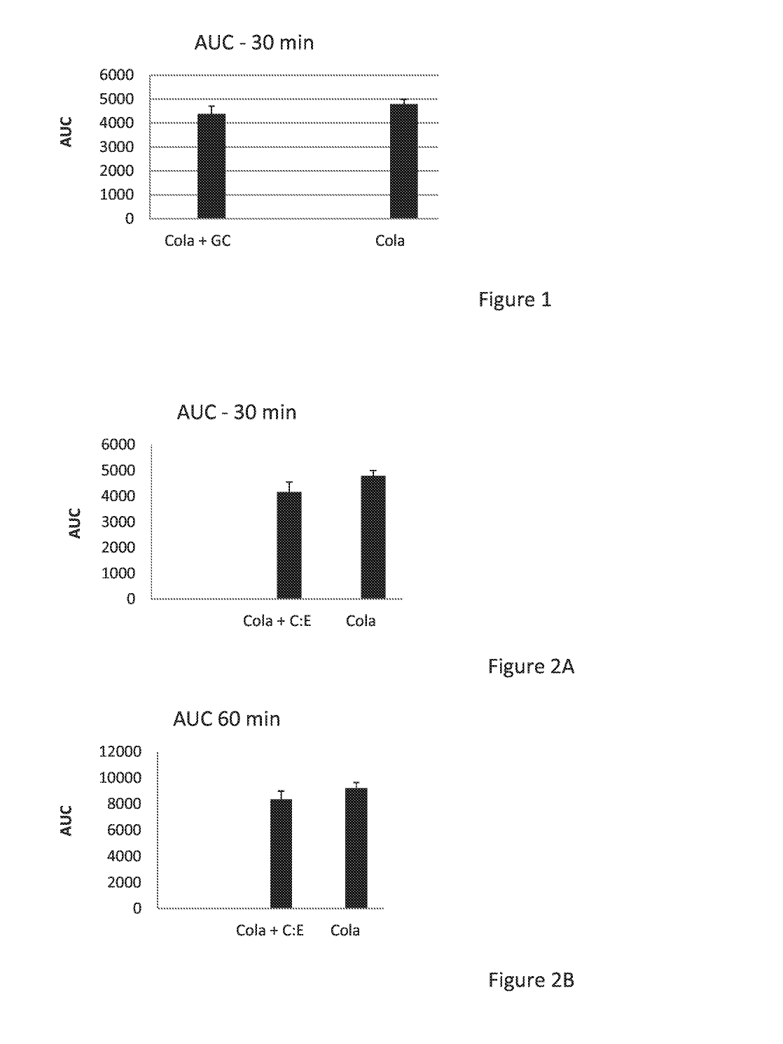

The data in Table 1 and illustration thereof in FIG. 1 clearly show that add-on of the GC+C:E combination to Coca Cola attenuate the elevation in blood sugar levels occurring at 30 min after consumption of SSB (p=0.03 between groups).

To further evaluate the effect of C:E on blood sugar levels following consumption of Coca Cola, the inventors performed the experiment in Table 2, including group A receiving orally Coca Cola +C:E (350 μl/mouse by gavage) and group B receiving Coca Cola only (300 μl/mouse by gavage). Glucose levels were measured at 0, 15, and 30 min after Coca Cola administration.

TABLE 2
The effect of C:E on blood sugar levels after consumption of Coca Cola

| Group | Treatment | AUC 60 minutes | AUC 30 minutes |
|---|---|---|---|
| A | Coca Cola + CE | 8342.5 ± 648 | 4160 ± 395 |
| N = 6 | | | |
| B | Coca Cola alone | 9195 + 441 | 4785 ± 208 |
| N = 6 | | | |

Table 2 and FIGS. 2A and 2B illustrate that add-on of C:E to Coca Cola attenuates the elevation in blood sugar levels at 30 and 60 min after consumption of SSB (p=0.01 and p=0.02 between groups for AUG at 30 min. and 60 min., respectively).

### Example 2

### GC and CE Add-on to Orange Flavored Soda Prevents Increase of Sugar Levels in the Serum

The inventors further examined the effect of the GC+C:E combination on additional soft drinks. Table 3 shows the experimental groups of mice, including group A receiving orally Soda Stream supplemented with GC with C:E (350 μl/mouse by gavage) and group B receiving—Soda Stream alone (300 μl/mouse by gavage). Glucose levels were measured at 0, 15, and 30 and 60 min after Soda Stream administration.

TABLE 3
The effect of GC + C:E on blood sugar levels
after consumption of Soda Stream

| Group | Treatment | AUC 30 minutes | AUC 60 minutes |
|---|---|---|---|
| A | Soda Stream + GC with | 3406 ± 440 | 7073.75 ± 675 |
| N = 6 | C:E | | |
| B | Soda Stream | 4121 ± 293 | 8176.25 ± 364 |
| N = 6 | | | |

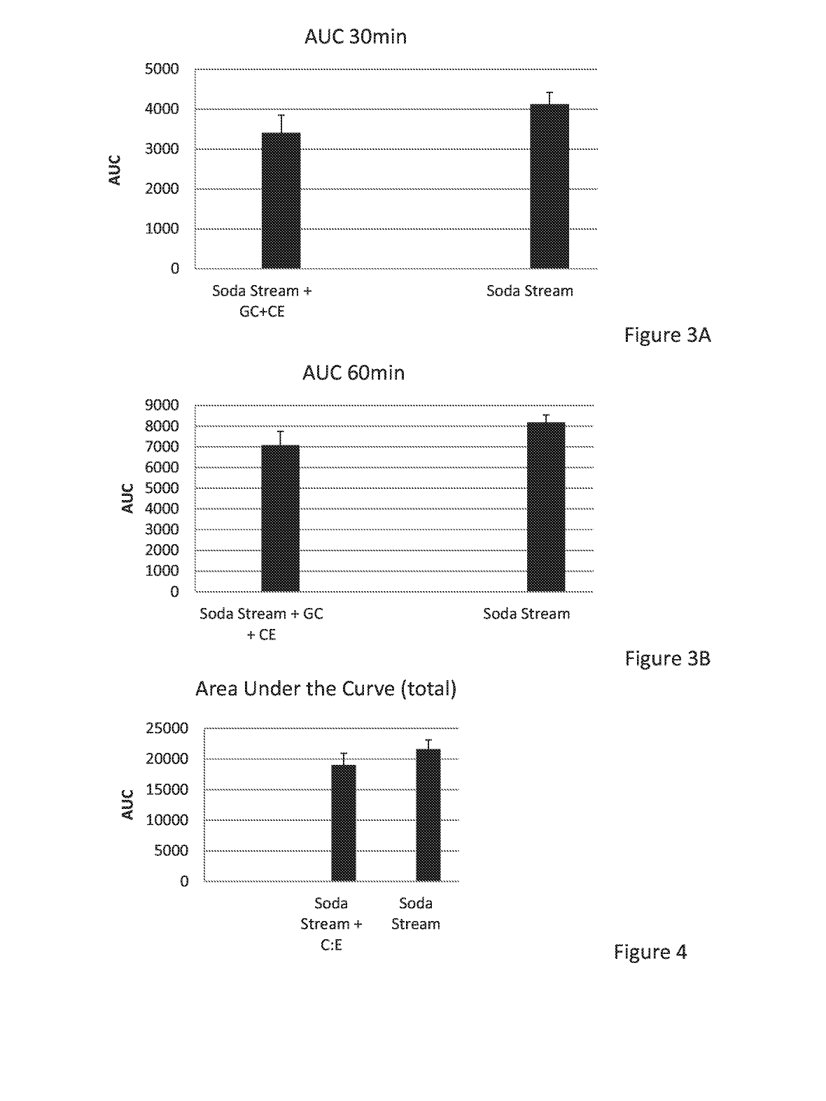

FIGS. 3A and 3B illustrate that add-on of the GC+C:E combination to Soda Stream prevents the elevation of blood sugar levels accompanying consumption of SSBs (p=0.01 between groups for both, AUC 30 min. and AUC 60 min.). As shown in FIG. 3B, the effect persists after 60 min.

To evaluate the effect of C:E alone an experiment as illustrated in Table 4 was performed, including group A receiving orally Soda Stream+C:E (350 μl/mouse by gavage) and group B receiving Soda Stream alone (300 μl/mouse by gavage). Glucose levels were measured at 0, 180 min after Soda Stream administration.

TABLE 4
The effect of C:E on blood sugar levels after consumption of Soda Stream

| Group | Treatment | AUC 180 minutes |
|---|---|---|
| A | Soda Stream + CE | 19033.5 ± 1920 |
| N = 6 | | |
| B | Soda Stream | 21591.25 ± 1533 |
| N = 6 | | |

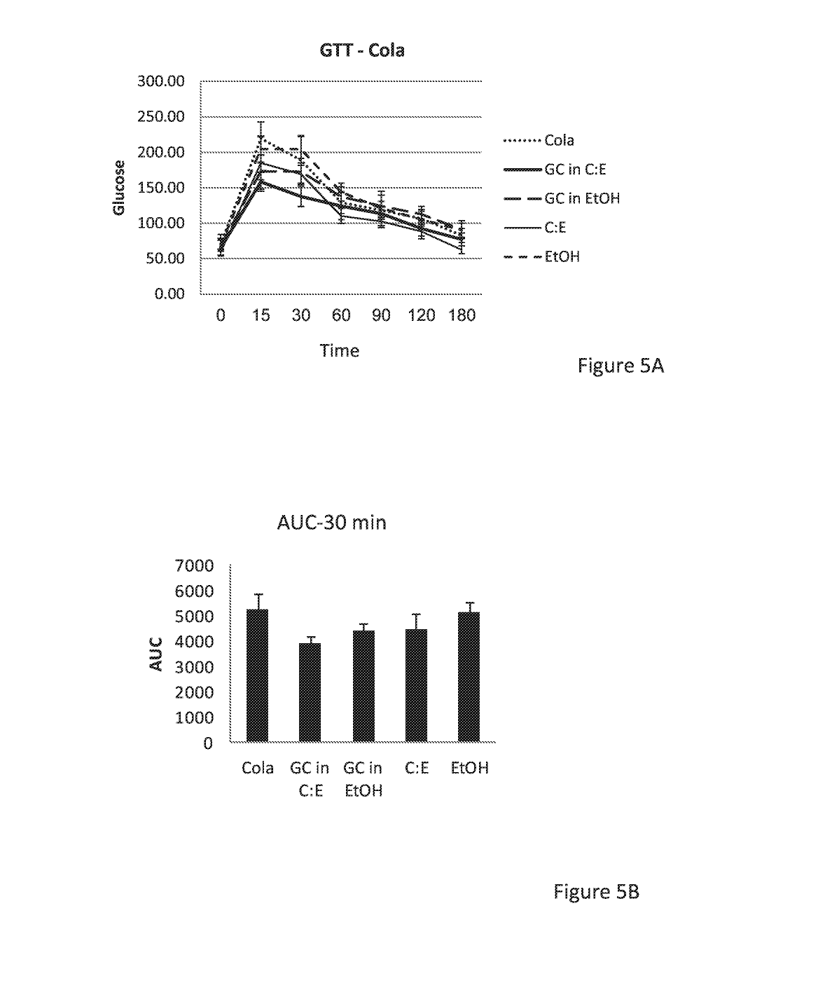

The results in Table 4 and FIG. 4 show that add-on of C:E to Soda Stream attenuates the elevation in sugar levels and this effect could be evident r 180 min. after SSB consumption (p=0.04 between the two groups).

### Example 3

### Combination of GC with CE Add-on to SSBs Exerts a Synergistic Effect in Attenuating the Increase in Serum Sugar Levels

The inventors further characterized the ameliorating effect of the GC−C:E combination on the increase in blood sugar caused by SSBs Table 5 shows the experimental groups of mice, including group A receiving Coca Cola, group B—GC in C:E (Cremophor: Ethanol in 1:1 ratio), group C—Coca Cola with GC in ethanol, group D—Coca cola with C:E and Group E—Coca Cola with ethanol. The glucose blood levels of animals of the different groups were assayed in a glucose tolerance test (GTT).

TABLE 5
The effect of GC + C:E on blood sugar levels
after consumption of Coca Cola

| Group | Treatment |
|---|---|
| A | Coca Cola |
| N = 6 | |
| B | Coca Cola + GC in C:E |
| N = 6 | |
| C | Coca Cola + GC in Ethanol |
| N = 6 | |
| D | Coca Cola + C:E |
| N = 6 | |
| E | Coca Cola + Ethanol |
| N = 6 | |

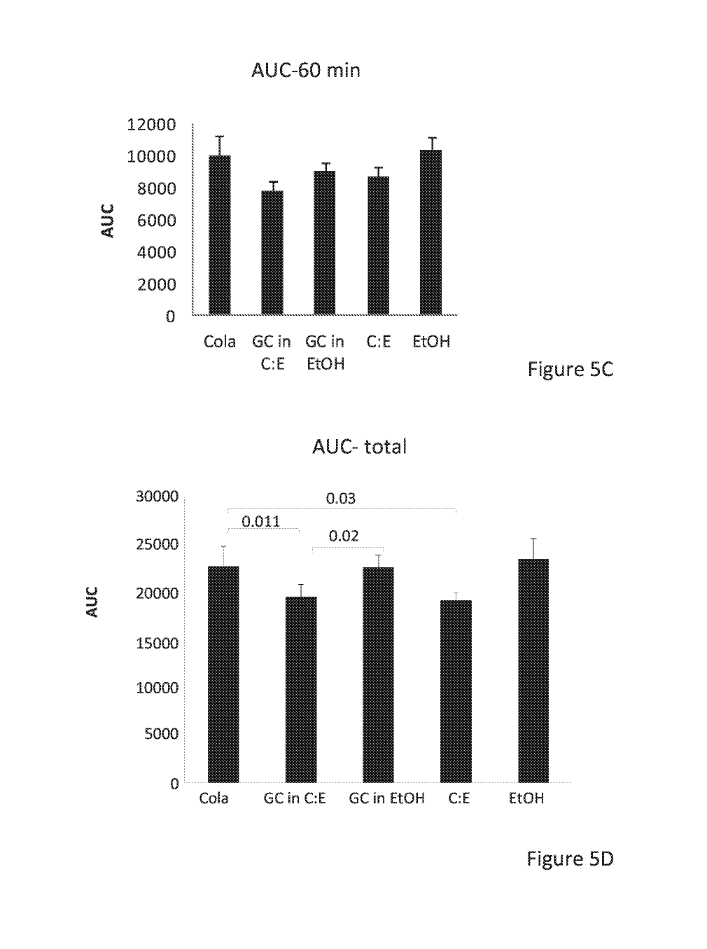

FIGS. 5A-5D show the effect of GC+C:E and C:E on GTT. More specifically, FIG. 5A shows that GC+C:E improve GTT as compared to controls. FIG. 5B shows AUC at 30 minutes whereby effects of GC in EtOH and GC+CE were significant (p=0.012 and p=0.01, respectively, compared to controls) and CE alone was not significant. Further, GC+CE had more significant effect compared to GC in ethanol (p=0.011). Further in FIG. 5C at 60 minutes, GC+CE exerted a synergistic effect compared to CE alone (p=0.038) and controls (p=0.003). In these conditions CE alone had an effect on GTT (p=0.004). In summary, as evident in FIG. 5D, the combination of GC with CE clearly demonstrated more significant effect on GTT compared to CE alone. (p=0.011, 0.03, respectively, compared to controls). Furthermore, the effect of GC+CE combination was synergistic compared to GE alone (p=0.02).

### Example 4

### A Synergistic Effect of GC with CE in Alleviating the Immune-Mediated Liver Damage in the Concanavalin a Hepatitis Model

The inventors next examined the effect of the combination of GC and C:E on the immune-mediated hepatitis in Con A mouse model. Details on the relevant experimental procedures are given above in experimental procedures. In brief, mice were injected i.v. with 250 μl ConA (0.5 mg/mouse) for 15 hrs prior to oral treatments, including C:E: ethanol 1:1 solution administered (30 μl/mouse); GC with CE as 100 mg GC dissolved in 1 ml C:E or GC in ethanol as 100 mg GC dissolved in 1 ml ethanol abs. administered (3 mg/mouse); control as PBS administered (30 μl/mouse). All mice were sacrificed 15 hours following ConA administration and serum levels of two liver enzymes were measured, ALT and AST Table 6 shows the experimental groups and the serum ALT and AST levels.

TABLE 6
The effect of GC + C:E on the serum ALT and AST levels in ConA model

| | ConA | Treatment | ALT | AST |
|---|---|---|---|---|
| Control | 0.5 mg/mouse | — | 8887 | 8807 |
| N = 4 | | | | |
| N = 5 | 0.5 mg/mouse | GC in C:E | 872 | 1043 |
| N = 4 | 0.5 mg/mouse | GC in EtOH | 1875 | 2231 |
| N = 5 | 0.5 mg/mouse | C:E | 1472 | 1407 |

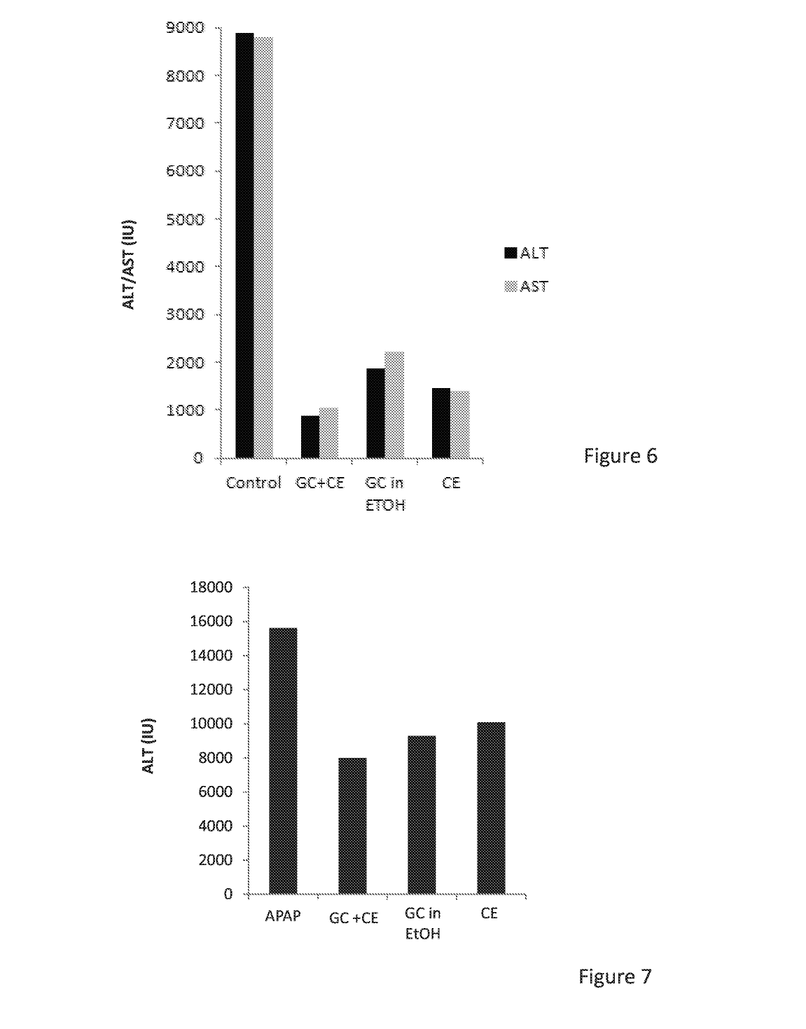

FIG. 6 shows that the combination of GC with CE had significant effects in alleviating the immune mediated liver damage, as reflected by reduction in AST and ALT levels, compared to each of the compounds alone (p<0.005).

In summary, as the combination of the invention was capable of inducing significant ameliorating effects on liver damage, it suggests that in addition to controlling blood sugar it is further a liver protecting combination. As this model is a model of immune mediated cytokine storm, it shows that GC+CE exerts a profound synergistic anti inflammatory effect and thus can serve as an anti inflammatory and immunomodulatory agent.

### Example 5

### A Synergistic Effect of GC with CE in Alleviating Drug-Mediated Liver Toxicity Induced by Acetaminophen (APAP)

Following from the above findings, the inventors further examined the effect of the combination of GC and C:E on drug-mediated liver toxicity, specifically toxicity resulting from previous exposure to acetaminophen (APAP also paracetamol).

APAP-mediated liver toxicity was induced in male adult mice fasted overnight and 2 hours after APAP administration. APAP was i.v. injected at the concentration of 4.6 mg/mouse. Various treatments, GC or C:E alone, GC with C:E and controls, were administered 1 hour after APAP injection. For various oral treatments, C:E: Cremophor:ethanol 1:1 solution was administered (30 μl/mouse); GC with CE: 100 mg GC dissolved in 1 ml C:E or GC in ethanol: 100 mg GC dissolved in 1 ml Ethanol Abs. were administered (3 mg/mouse); Control:PBS administered (30 μl/mouse). Table 7 shows the experimental groups and the serum ALT levels measured.

TABLE 7
The effect of GC + C:E on the serum ALT levels in APAP model

| | APAP | Treatment | ALT |
|---|---|---|---|
| Control | 4.6 mg/mouse | — | 15612 |
| N = 6 | | | |
| B | 4.6 mg/mouse | GC in C:E | 7996 |
| N = 6 | | | |
| C | 4.6 mg/mouse | GC | 9290 |
| N = 6 | | | |
| D | 4.6 mg/mouse | C:E | 10085 |
| N = 6 | | | |

All mice were sacrificed 24 hours after APAP administration and serum levels of ALT were measured. FIG. 7 shows that the effect of the combination of GC with C:E was significantly more effective than the effect of GC or C:E alone in alleviating APAP-mediated liver damage, as reflected by reduction in ALT levels (p<0.005).

### Example 6

### The Effect of Oral Administration of GC with CE on Blood Sugar Levels in Humans

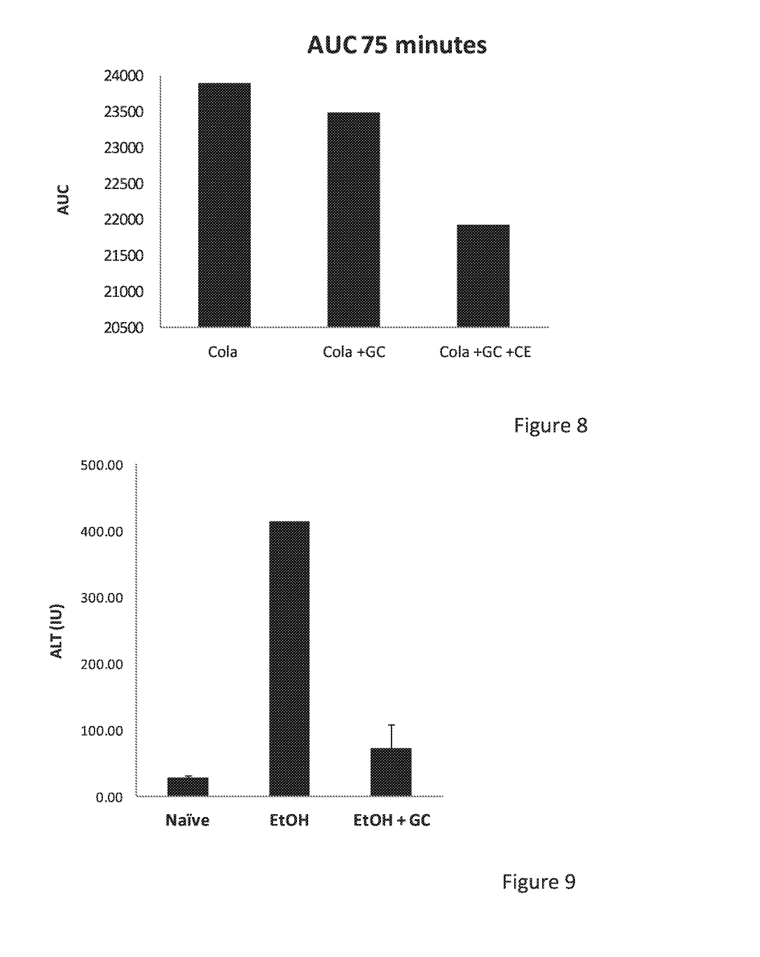

A human subject 56 year old healthy male was monitored for blood sugar levels for 2 hours every 15 minutes following an overnight fast and drinking of 500 ml of Coca Cola alone or Coca Cola with GC or GC with CE add-ons. Table 8 summarizes the blood sugar levels at the measured intervals and FIG. 8 shows the AUC values at 75 min.

TABLE 8
Glucose levels following oral administration of Coca Cola with GC

| Time (minutes) | | | | | | | | |
|---|---|---|---|---|---|---|---|---|
| 0 | 15 | 30 | 45 | 60 | 75 | 90 | 105 | 120 |

SSB ke only

| | | | | | | | | | |
|---|---|---|---|---|---|---|---|---|---|
| Glucose | 101 | 137 | 129 | 153 | 148 | 128 | 113 | 95 | 95 |
| (mg %) | | | | | | | | | |

SSB + 28 mg GC in ETOH

| | | | | | | | | | |
|---|---|---|---|---|---|---|---|---|---|
| Glucose | 95 | 128 | 135 | 152 | 132 | 124 | 124 | 96 | 96 |
| (mg %) | | | | | | | | | |

SSB + 28 mg GC + CE

| | | | | | | | | | |
|---|---|---|---|---|---|---|---|---|---|
| Glucose | 86 | 151 | 155 | 148 | 112 | 111 | 91 | 91 | 91 |
| (mg %) | | | | | | | | | |

In summary, the above results clearly demonstrate that add-on the combination of GC with CE to SSB had a significant synergistic effect on controlling blood sugar levels in humans.

### Example 7

### Hepatoprotective Effect of GC with CE in on the Alcohol-Induced Liver Damage

The inventors further characterized the ameliorating effect of the GC—the alcohol induced liver damage in a mouse model. Table 9 summarizes the relevant experimental groups, including group A of naïve mice; group B receiving alcohol 6 gr/kg-300 μl of 70% Ethanol per mouse/gavage i.e. 3500 μl EtOH Abs.+1500 μl sterile water (Ethanol is equal to 6 gr/kg for 27.5 gr mouse body weight).; Group C receiving 6 microgram of GC with alcohol. Mice were sacrificed after 16 hours and the level of liver enzymes was evaluated.

TABLE 9
The effect of GC + C:E on the alcohol induced liver
damage in a mouse model

| Group | Treatment | ALT levees |
|---|---|---|
| A | naive mice | 28 |
| N = 4 | | |
| B | EtOH | 414 |
| N = 4 | | |
| C | EtOH + GC 6 microgram | 73 |
| N = 4 | | |

FIG. 9 shows that by the levels of liver enzymes, co-administration of GC with alcohol are protective for the alcohol-mediated liver damage.

### Example 8

### GC with CE Add-Ons to Sugar-Enriched Chocolate Milk Exerts a Synergistic Effect in Ameliorating the Increase in Serum Sugar Levels

The inventors further characterized the effects of GC with CE combination on the increase in blood sugar caused by sugar-enriched chocolate milk Table 10 shows the experimental groups of mice, including group A receiving chocolate milk (350 μl/mouse by gavage); group B—chocolate milk+CE; group C—chocolate milk with GC; and group D—chocolate milk+GC+CE. The glucose blood levels of animals of the different groups were assayed in a glucose tolerance test (GTT). Glucose levels were measured at 0, 15, 30, 60 and 90 min after chocolate milk administration.

TABLE 10
The effect of GC + C:E on blood sugar levels
after consumption of chocolate milk

| Group | Treatment |
|---|---|
| A | Chocolate milk |
| N = 2 | |
| B | Chocolate milk + CE |
| N = 2 | |
| C | Chocolate milk + GC |
| N = 2 | |
| D | Chocolate milk + GC + CE |
| N = 2 | |

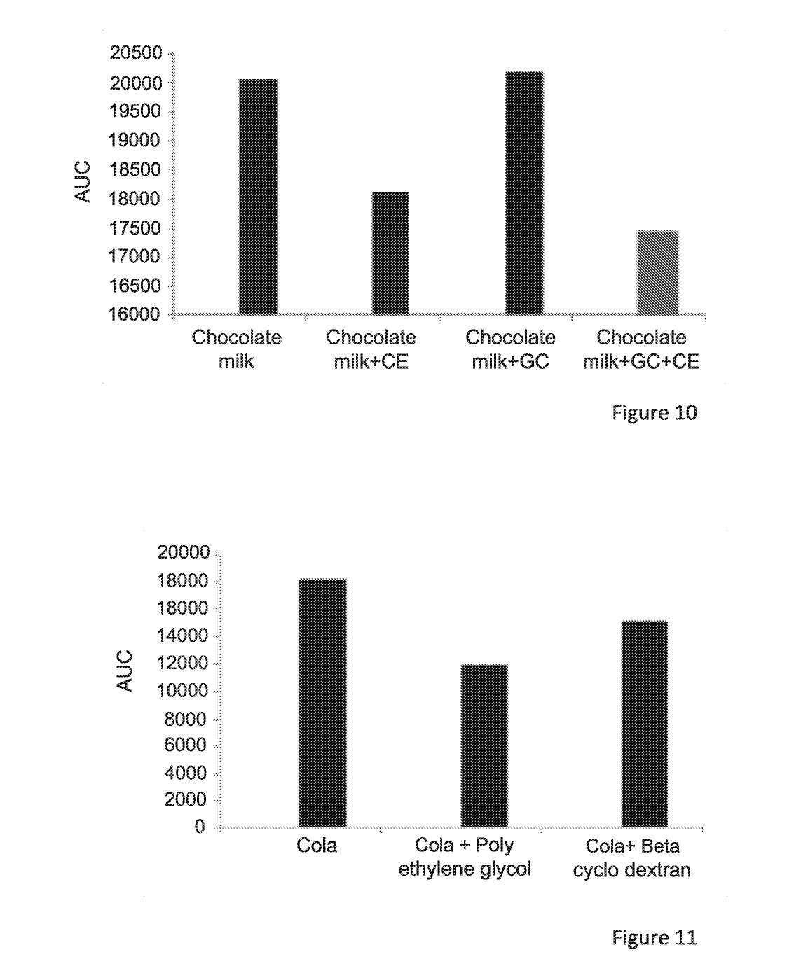

FIG. 10 shows the AUC values at 90 min, whereby GC+CE exerted a synergistic effect as compared to CE alone and controls. In these conditions CE alone also had an effect on GTT.

### Example 9

### A Synergistic Effect of GC with Polyethylene Glycol or with Beta Cyclodextrin Add-Ons to SSBS in Preventing the Increase in Serum Sugar Levels

The inventors further explored the effect of GC-in combination with polyethylene glycol or with beta cyclo dextrin on blood sugar levels caused by sugar-enriched soft drinks such as Coca Cola. Table 11 shows the experimental groups of mice, including group A receiving Coca Cola (350 μl/mouse by gavage); group B—GC with polyethylene glycol; group C—Coca Cola with GC in beta cyclo dextrin. The glucose blood levels were assayed in a glucose tolerance test (GTT) as above. Total AUC for the whole study was calculated per group.

TABLE 11
The effect of GC and other adjuvants on blood sugar levels
after consumption of Coca Cola

| Group | Treatment | Total AUC |
|---|---|---|
| A | Coca Cola | 18168 |
| N = 2 | | |
| B | Coca Cola + GC in polyethylene | 11925 |
| N = 2 | glycol | |
| C | Coca Cola + GC in beta cyclo | 15157 |
| N = 2 | dextrin | |

Table 11 and illustration thereof in FIG. 11 clearly show that the total AUC values were reduced in mice receiving GC+ with polyethylene glycol or with beta cyclo dextrin add-ons compared to controls, suggesting their potential applicability of these combinations for controlling blood sugar levels after SSBs consumption.

1. A combined composition for use in at least one of, a method for controlling blood sugar levels in a subject, a method for the treatment of an immune related disorder, a method of treating liver damage and a method for treating, preventing, ameliorating, reducing or delaying the onset of acute or chronic toxic effect of a drug, said composition comprising as an active ingredient at least one of:
(a) at least one natural or synthetic beta-glycolipid or any derivatives thereof;
(b) at least one polyethoxylated castor oil or any derivative thereof;
(c) at least one adjuvant selected from polyethylene glycol or beta cyclo dextrin or any derivative thereof; and
(d) any combination of (a), (b) and (c).

2. The composition according to claim 1, wherein said natural or synthetic beta-glycolipid is any one of a glucosylceramide, glycosphingolipid, monosaccharide ceramide, galatosylceremide, lactosylceramide, gal-gal-glucosyl-ceramide, GM2 ganglioside, GM3 ganglioside, globoside or any soy derivative or a combination thereof.

3. The composition according to claim 2, wherein said glucosylceramide is a beta glucosylceramide (GC).

4. The composition according to claim 1, wherein said derivative of polyethoxylated castor oil is Cremophore EL.

5. The composition according to claim 1, in a formulation adapted for add-on to a solid, semi-solid or liquid food, beverage, food additive, food supplement, medical food, drug and/or any type of pharmaceutical compound.

6. The composition according to claim 5, wherein said food and/or beverage comprise an increased content of sugar and alcohol or are associated with increase in blood sugar or alcohol level.

7. The composition according to any one of claims 1 and 6, for controlling blood sugar levels in a subject, wherein said control is inhibiting increase or decrease in blood sugar levels or altering the insulin resistance state in said subject.

8. The composition according to claim 7, for use in the prevention or alleviation of symptoms related to a condition associated with increased or decreased blood sugar levels, wherein said condition is any one of a hepatic disorder, pancreatic dysfunction, diabetes, obesity, weight gain, alcohol intoxication, alcohol withdrawal and vertigo, any condition associated with alteration of pancreatic or liver function or tissue or organ damage.

9. The pharmaceutical composition according to claim 7, for use in a method for treating a subject suffering from a disorder associated with increased or decreased blood sugar levels.

10. The composition according to claim 9, wherein said disorder is any one of a hepatic disorder, pancreatic dysfunction, diabetes, obesity, insulin resistance, metabolic syndrome, alcohol intoxication, alcohol withdrawal and vertigo, an inflammation of pancreas, liver, muscle or the adipose tissue.

11. The composition according to claim 10, optionally further comprising additional therapeutic agent, wherein said additional therapeutic agent is any one of insulin, N-acetyl cysteine (NAC), thiamine (vitamin B1), a benzodiazepine or any combination thereof.

12. The composition according to any one of claims 1-4, for use in a method for treating, preventing, ameliorating, reducing or delaying the onset of an immune-related disorder.

13. The composition according to claim 12, wherein said immune-related disorder is any one of an inflammatory disorder, an autoimmune disorder, an infectious disease and a proliferative disorder.

14. The composition according to claim 13, wherein said immune-related disorder is hepatitis.

15. The composition according to claim 12, wherein said composition further comprises at least one additional therapeutic agent.

16. The composition according to any one of claims 1-4, for use in a method for treating liver damage in a subject in need thereof, said composition comprising a therapeutically effective amount of a natural or synthetic beta-glycolipid and polyethoxylated castor oil or any derivative thereof, or any combination thereof.

17. The composition according to claim 16, wherein said subject is suffering from a liver disease, said liver disease is any one of viral, alcoholic or autoimmune hepatitis, alcoholic or autoimmune cirrhosis, alcoholic fatty liver disease, non alcoholic fatty liver disease (NAFLD), liver steatosis, alcoholic or nonalcoholic steatohepatits (NASH), hepatocellular carcinoma, drug-induced liver disease and pediatric liver disease and metabolic liver disease.

18. The pharmaceutical composition according to claim 17, wherein said composition optionally further comprising at least one additional therapeutic agent, said additional therapeutic agent is any one of insulin, NAC, vitamin B1, a benzodiazepine, an anti-viral or anti-inflammatory drug, antibody, a chemotherapeutic agent and a gut hormone.

19. The composition according to any one of claims 1-4, for use in a method for treating, preventing, ameliorating, reducing or delaying the onset of acute or chronic toxic effect of a drug.

20. The composition according to claim 19, wherein said drug is an analgesic or an antipyretic drug.

21. A method for controlling blood sugar levels in a subject, treating an immune related disorder, treating liver damage and for treating, preventing, ameliorating, reducing or delaying the onset of acute or chronic toxic effect of a drug, the method comprising providing to a subject a food supplement comprising as an active ingredient at least one of:
(a) at least one a natural or synthetic beta-glycolipid;
(b) at least one polyethoxylated castor oil or any derivative thereof;
(c) at least one adjuvant selected from polyethylene glycol or beta cyclo dextrin or any derivative thereof; and
(d) any combination of (a), (b) and (c); and
(e) a composition comprising any one of (a), (b), (c) and (d).

22. The method according to claim 21, wherein said natural or synthetic beta-glycolipid is any one of a glucosylceramide, glycosphingolipid, monosaccharide ceramide, galatosylceremide, lactosylceramide, gal-gal-glucosyl-ceramide, GM2 ganglioside, GM3 ganglioside, globoside or any soy derivative or a combination thereof.

23. The method according to claim 22, wherein said glucosylceramide is a beta glucosylceramide (GC).

24. The method according to claim 21, wherein said derivative of polyethoxylated castor oil is Cremophore EL or any derivative thereof.

25. The method according to claim 21, wherein said composition is in a formulation adapted for add-on to a solid, semi-solid or liquid food, beverage and/or drug.

26. The method according to claim 25, wherein said food and/or beverage comprises an increased content of sugar and/or alcohol.

27. The method according to any one of claims 21 and 26, for controlling blood sugar levels in a subject, wherein said control is inhibiting increase or decrease in blood sugar levels.

28. The method according to claim 27, for the prevention or alleviation of symptoms related to a condition associated with increased or decreased blood sugar levels, wherein said condition is any one of a hepatic disorder, pancreatic dysfunction, diabetes, obesity, weight gain, alcohol intoxication, alcohol withdrawal, vertigo, and tissue or organ damage.

29. The method according to claim 27, for treating a subject suffering from a disorder associated with increased or decreased blood sugar levels.

30. The method according to claim 29, wherein said disorder is any one of a hepatic disorder, pancreatic dysfunction, diabetes, obesity, insulin resistance, metabolic syndrome, alcohol intoxication, alcohol withdrawal, vertigo, and tissue or organ damage.

31. The method according to claim 21, optionally further comprising the concurrent or parallel administration of an additional therapeutic agent, wherein said additional therapeutic agent is any one of insulin, N-acetyl cysteine (NAC), thiamine (vitamin B1), a benzodiazepine, antibodies, gut hormones or any combination thereof.

32. The method according to any one of claims 21-24, for treating, preventing, ameliorating, reducing or delaying the onset of an immune-related disorder.

33. The method according to claim 32, wherein said immune-related disorder is any one of an inflammatory disorder, an autoimmune disorder, an infectious disease and a proliferative disorder.

34. The method according to claim 33, wherein said immune-related disorder is hepatitis.

35. The method according to claim 32, wherein said method further comprises the concurrent or parallel administration of at least one additional therapeutic agent.

36. The method according to any one of claims 21-24, for treating liver damage in a subject in need thereof, said composition comprising a therapeutically effective amount of a natural or synthetic beta-glycolipid and polyethoxylated castor oil or any adjuvant selected from polyethylene glycol and beta cyclo dextrin or any derivative thereof, or any combination thereof.

37. The method according to claim 36, wherein said subject is suffering from a liver disease, said liver disease is any one of viral, alcoholic or autoimmune hepatitis, alcoholic or autoimmune cirrhosis, alcoholic fatty liver disease, non alcoholic fatty liver disease (NAFLD), any type of liver steatosis due to other disease such as Wilson's disease or alpha 1 anti trypsin deficiency, alcoholic or nonalcoholic steatohepatits (NASH), hepatocellular carcinoma, drug-induced liver disease and pediatric liver disease, any type of metabolic liver disease such as glycogen storage disease pediatric liver disease

38. The method according to claim 37, further comprising the concurrent or parallel administration of at least one additional therapeutic agent, said additional therapeutic agent is any one of insulin, NAC, vitamin B1, a benzodiazepine, an anti-viral or anti-inflammatory drug, antibody, gut hormones and a chemotherapeutic agent.

39. The method according to any one of claims 21-24, for treating, preventing, ameliorating, reducing or delaying the onset of acute or chronic toxic effect of a drug.

40. The method according to claim 39, wherein said drug is an analgesic or an antipyretic drug.

41. A pharmaceutical composition for use in a method for treating liver damage in a subject in need thereof, said composition comprising as an active ingredient a therapeutically effective amount of a polyethoxylated castor oil or an adjuvant selected from polyethylene glycol and beta cyclo dextrin or any derivative or a combination thereof, and optionally further comprising a pharmaceutically acceptable carrier.

42. A soft or an alcoholic beverage comprising at least one polyethoxylated castor oil or any derivative and at least one a natural or synthetic beta-glycolipid.

43. A combined composition comprising as an active ingredient at least one natural or synthetic beta-glycolipid and at least one polyethoxylated castor oil or adjuvant selected from polyethylene glycol and beta cyclo dextrin or any derivative thereof.

44. A pharmaceutical composition for use in a method for prevention of liver steatosis or liver disease in a healthy subject exposed to conditions inducing a liver disease, said composition comprising as an active ingredient a therapeutically effective amount of a polyethoxylated castor oil or an adjuvant selected from polyethylene glycol and beta cyclo dextrin or any derivative or a combination thereof, and optionally further comprising a pharmaceutically acceptable carrier.

45. A pharmaceutical composition for use in a method for prevention of diabetes in a subject with pre diabetes condition, said composition comprising as an active ingredient a therapeutically effective amount of a polyethoxylated castor oil or polyethylene glycol or with beta cyclo dextrin or any derivative or a combination thereof, and optionally further comprising a pharmaceutically acceptable carrier.
